(19) United States
(12) Patent Application Publication
(10) Pub. No.: US 2024/0229089 A1
(43) Pub. Date: Jul. 11, 2024

(54) A process for enzymatic synthesis of amides from amines and carboxylic acids or esters

(71) Applicant: XP Chemistries AB, Sundsvall (SE)

(72) Inventors: Shuangzheng Lin, Solna (SE); Armando Cordova, Stockholm (SE); Luca Deiana, Sundsvall (SE); Ismail Ibrahem, Sundsvall (SE)

(73) Assignee: XP Chemistries AB, Sundsvall (SE)

(21) Appl. No.: 18/555,537

(22) PCT Filed: Apr. 28, 2022
(86) PCT No.: PCT/EP2022/061321

§ 371 Date: Oct. 15, 2023

(30) Foreign Application Priority Data
Apr. 30, 2021 (SE) 2150548-2

(51) Int. Cl. C12P 13/02

(52) U.S. Cl. CPC C12P 13/02

(57) ABSTRACT

The present invention relates to A process for enzymatic synthesis of amides of formula (III) from amines of formula (I) and compounds of formula (II). characterized in that the lipase is immobilized on a rotary bed reactor or on a spin-fixed-bed reactor and a Dean-Stark apparatus is used for dehydration.

### FIELD OF THE INVENTION

The present invention relates to a process for enzymatic synthesis of amides from amines and carboxylic acids or esters using a lipase.

### BACKGROUND OF THE INVENTION AND PRIOR ART

Amide linkage is important in development of numerous compounds, such a pharmaceutical drugs and polymers. Several processes for direct catalytic amidation have been developed over the years.

In thermal amidation, no catalyst may be used. This process is performed at high temperature (>140° C.) and the yield is dependent on the temperature used, the concentration of the substrate, the solvent used and other parameters.

Metal-based amidations have been done using boron-based catalysts or palladium-based catalyst. Although higher yield can be obtained compared to thermal amidation, the processes are expensive and time consuming. Recycling of catalysts and solvents is challenging.

Neither thermal nor metal based amidations are environmentally friendly processes. Several attempts have been made to improve the efficiencies of the processes and reduce the costs and carbon food-print.

In amidation processes, water must be removed to improve the yield of the processes. Most of amidation processes are therefore performed under reduced pressure. This increases costs and thus increases difficulties for large scale amidation. Molecular sieves may be used as well, but these are still expensive for use at large scale. A Dean-Stark apparatus may be used as well to remove water from an amidation process.

Enzymatic amidation has been developed over the years using different kind of enzymes like lipases. These so-called biocatalysts can be used at lower temperature and show good selectivity. However, current technologies show very limited substrate scope and often require long reaction times (days). Combining enzymatic amidation with a palladium catalyst may result in a yield of about 70% as shown by Palo-Nieto et al., ACS Catal., 2016, 6, 3932-3940.

Another drawback of biocatalysts is costs. To reduce the costs and improve efficiency of the amidation process, the enzymes can be immobilized e.g. on beads during the reaction. This allows recirculation of the enzyme. The use of flow reactors has further improved the biocatalytic amidation process. However, recirculation of the lipase is both time- and cost-ineffective.

Up to date, there is no environmentally friendly catalytic amidation process that is sufficiently efficient and cost effective to be used for large scale production. This is a top priority of the American Chemical Society Green Chemistry Pharmaceutical Roundtable (https://www.acsgcipr.org). Today, most methods utilize stoichiometric amounts of toxic activating reagents, Dunetz et. al. Org. Process. Res. Dev. 2016, 20, 140. Thus, there is still a need for a greener and more cost effective amidation process that can be used at a larger scale.

Capsaicinoids are commonly used in food environmentally friendly products. Capsaicin is also widely used in the pharmaceutical industry. Capsaicin is for example used as an analgesic in topical ointments and dermal patches to relieve minor aches and pains of muscles and joints associated with arthritis, backache, strains and sprains, or to reduce the symptoms of peripheral neuropathy.

Capsaicinoids can be isolated from natural sources (e.g. Capsicum spp pepper fruits), but this gives predominantly capsaicin and dihydrocapsaicin, since many of the other capsaicinoids are present only in trace amounts. Chemical synthesis is thus useful to obtain the more uncommon capsaicinoids, such as nonivamide, and for making none-natural capsaicinoids. Capsaicinoids can be prepared from vanillin by first reducing vanillin oxime using a mixture of an excess of metal (Zn) and ammonium formate in methanol under reflux to obtain vanillylamine. Alternatively, the amide bond-formation can be accomplished by an enzyme-catalyzed transformation between vanillylamine and different fatty acid derivatives. WO2015/144902A1 discloses a multi-catalytic cascade relay sequence involving an enzyme cascade system that when integrated with other catalytic systems, such as heterogeneous metal catalysts and organic catalysts, converts an alcohol to an amine and amide in sequence or in one-pot.

US2017081277A1 discloses an amidation using dialkyl-amines as substrates. Novozym 435™ immobilized on beads are used. A Dean Stark apparatus may be used to remove ethanol from the reaction mixture. The reactions are performed under reduced pressure. For large scale manufacturing, beads are not suitable because it is costly and time consuming to separate the beads from the reaction mixture. Further, for large scale production, reduced pressure is preferably avoided to reduce cost and time of the overall process.

U.S. Pat. No. 6,022,718 discloses a process for preparation of capsaicin analogues using hydrolysis and capsaicin as starting materials.

Pithani S., Using spinchem rotation bed reactor technology for immobilized enzymatic reactions: a case study, Org. Process Res. Dev., 2019, vol.23, pages 1926-1931, discloses advantages of using rotary bed immobilized lipase. An acylation reaction is used to demonstrate that lipase (Novozym 435™) can be used in a rotating bed reactor. The loading was limited to 10 wt % due to high costs. A loading of 5 to 10 wt % was deemed sufficient to achieve a conversion of 45-50% within 6 hours. The overall yield after upscaling was 39%. Although Pithani shows that a rotation bed reactor is useful for acylations, it also shows that it is costly and results in a conversion of 45-50% with an overall yield of 39%. The results disclosed in Pithani are disconcerting for large scale production using a rotation bed reactor.

There is an increasing need for large scale production of amide compounds like capsaicinoids. Such processes are preferably efficient and effective having improved yields compared to known processes. Such amidation processes are preferably environmentally friendly and especially cost effective.

### SUMMARY OF THE INVENTION

It is an object of the present invention to at least partly overcome the above-mentioned problems, and to provide an improved process for the synthesis of amides from amines and carboxylic acids or esters.

This object is achieved by a process as defined in the claims.

One aspect relates to a process for enzymatic synthesis of amides of formula III from amines of formula I and compounds of formula II,

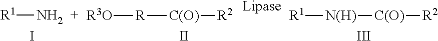

wherein R¹ is selected from the group comprising or consisting of C₁₋₁₂alkyl-, C₁₋₁₂alkenyl-, C₁₋₁₂alkynyl-, C₁₋₁₂alkoxy-, C₁₋₁₂alkyl-O-C₁₋₁₂alkyl-, C₁₋₁₂alkyl-OC(O)—C₁₋₁₂alkyl-, C₁₋₁₂alkyl-NH—C₁₋₁₂alkyl-, C_{1- 12}alkyl-NHC(O)—C₁₋₁₂alkyl-, C₃₋₁₂cycloalkyl-, C₃₋₁₂cycloalkenyl-, C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R¹ may optionally be substituted with one or more substituent selected from the group comprising or consisting of hydrogen, hydroxy, oxy, halogen, carboxy, amine, amide, C₁₋₆hydroxyalkyl-, C₁₋₆haloyalkyl-, C₁₋₆amineoxyalkyl-, C₁₋₆amideyalkyl-, C₁₋₆carboxyalkyl-, C₁₋₆sulfuralkyl-, C₁₋₆sulfidealkyl-and C₁₋₆alkoxy-, and wherein one or more carbon in a cycloalkyl, cycloalkenyl or aryl may be substituted with one or more heteroatoms selected from O, N or S, wherein R² is selected from the group comprising or consisting of hydrogen, C₁₋₃₀alkyl-, C₁₋₃₀alkenyl-, C₁₋₃₀alkynyl-, C₁₋₃₀alkoxy-, C₁₋₃₀alkyl-O—C₁₋₁₂alkyl-, C₁₋₃₀alkyl-OC(O)—C₁₋₁₂alkyl-, C₁₋₃₀alkyl-NH—C₁₋₁₂alkyl-, C₁₋₃₀alkyl-NHC(O)—C₁₋₁₂alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R² may optionally be substituted with one or more substituent selected from the group comprising or consisting of hydrogen, hydroxy, oxy, halogen, carboxy, amide, C₁₋₆hydroxyalkyl-, C₁₋₆haloyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S, wherein R³ is selected from the group comprising or consisting of hydrogen, C₁₋₆alkyl-, C₁₋₆alkenyl-, C₁₋₆alkynyl-, C₁₋₆alkoxy-, C₁₋₆alkyl-O—C₁₋₆alkyl-, C₁₋₆alkyl-OC(O)—C₁₋₆alkyl-, C₁₋₆alkyl-NH-C₁₋₆alkyl-, C₁₋₆alkyl-NHC(O)-C₁₋₆alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R³ may optionally be substituted with one or more substituent selected from the group comprising or consisting of hydrogen, hydroxy, oxy, halogen, carboxy, amine, amide, C₁₋₆hydroxyalkyl-, C₁₋₆haloyalkyl-, C₁₋₆amineoxyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S, and wherein R is a bond or C₁₋₆alkyl-, characterized in that the lipase is immobilized on a rotary bed reactor or on a spin-fixed-bed reactor and a Dean-Stark apparatus is used for dehydration.

In some aspects, lipase is immobilized on a rotary bed reactor and a Dean-Stark apparatus is used for dehydration.

In some aspects, a lipase immobilized on beads is disclaimed.

In the processes of the invention, as defined anywhere in here, a combination of enzyme catalysis and azeotropic dehydration is used for direct catalytic amide synthesis. The enzyme, lipase, is immobilized on a rotary bed reactor or on a spin-fixed-bed reactor. Compared to immobilizing lipase on beads or using sieves, the lipase in the process of the invention can easily be recirculated. This allows the process to be performed in a time- and cost-effective manner, especially at large scale.

The unique combination of a rotary bed reactor or a spin-fixed-bed reactor and a Dean-Stark apparatus improves the yield (>90, or 99%) as well as the conversion rate (>90 or 99%). The unique combination allows the use of wet raw material. The process can be performed at atmospheric pressure and at temperatures below 100° C. (60-90° C.). The process is environmentally friendly. The process is suitable for large scale production of amides.

An easy workup and purification process allow the process to be used at a large scale. The enzymes and the solvents used, if any, are easy to recycle, which in turn makes large scale production feasible. The unique combination of an immobilized enzyme on a rotary bed reactor or on a spin-fixed-bed reactor and a Dean Trap apparatus allows the process to be extended to the synthesis of other amides and esters.

In one aspect, the process is performed under neat conditions. The process can be performed without any solvent. This may improve the efficiency and effective and environmentally friendliness of the process. It also reduces costs for performing the process. A neat process further reduces costs for the process on a large scale.

Compared to known processes, the direct amidation process of the invention has an improved conversation rate as well as an improved yield. Less process steps are needed for the amidation, which reduces time and costs. The mass flow is improved in the process of the invention. Because the enzyme is immobilized/fixed, the reaction products can easily be filtered off and purified. The process of the invention has an improved reaction rate.

The process allows for effective and efficient large-scale production of amide compounds like capsaicinoids. The process has improved yields compared to known processes. The amidation processes are environmentally friendly and especially cost-effective.

The combined use of the rotary bed reactor and the Dean-Stark apparatus allows for control of the moisture content during the process. A low moisture content improves conversion rate and yield. The results in Cycle 2 of Table 1 in example 14, show that even raw material having a moisture content of 23wt % can be used. This improves the flexibility of the process. This also improves the feasibility for large scale use of the process.

In some aspects, the option for R² to be a (dialkyl)-amine is disclaimed.

According to some aspect of the invention, R¹ is selected from the group comprising or consisting of C₁₋₁₂alkyl-, C₁₋₁₂alkenyl-, C₁₋₁₂alkynyl-, C₁₋₁₂alkoxy-, C₁₋₁₂alkyl-O-C₁₋₁₂alkyl-, C₁₋₁₂alkyl-OC(O)-C₁₋₁₂alkyl-, C₃₋₁₂cycloalkyl-, C₃₋₁₂cycloalkenyl-, C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R¹ may optionally be substituted with one or more substituent selected from the group comprising or consisting of hydrogen, hydroxy, oxy, halogen, carboxy, C₁₋₆hydroxyalkyl-, C₁₋₆haloyalkyl-, C₁₋₆carboxyalkyl-, C₁₋₆sulfuralkyl-, C₁₋₆sulfidealkyl- and C₁₋₆alkoxy-, and wherein R² is selected from the group comprising or consisting of hydrogen, C₁₋₃₀alkyl-, C₁₋₃₀alkenyl-, C₁₋₃₀alkynyl-, C₁₋₃₀alkoxy-, C₁₋₃₀alkyl-O-C₁₋₁₂alkyl-, C₁₋₃₀alkyl-OC(O)-C₁₋₁₂alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R² may optionally be substituted with one or more substituent selected from the group comprising or consisting of hydrogen, hydroxy, oxy, halogen, carboxy, C₁₋₆hydroxyalkyl-, C₁₋₆haloyalkyl-, C₁₋₆carboxyalkyl-, C₁₋₆sulfuralkyl-, C₁₋₆sulfidealkyl- and C₁₋₆alkoxy-, and wherein R³ is selected from the group comprising or consisting of hydrogen, C₁₋₆alkyl-, C₁₋₆alkenyl-, C₁₋₆alkynyl-, C₁₋₆alkoxy-, C₁₋₆alkyl-O-C₁₋₆alkyl-, C₁₋₆alkyl-OC(O)-C₁₋₆alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R³ may optionally be substituted with one or more substituent selected from the group comprising or consisting of hydrogen, hydroxy, oxy, halogen, carboxy, C₁₋₆hydroxyalkyl-, C₁₋₆haloyalkyl-, C₁₋₆carboxyalkyl-, C₁₋₆sulfuralkyl-, C₁₋₆sulfidealkyl- and C₁₋₆alkoxy-, and wherein R is a bond or C₁₋₆alkyl-.

According to some aspect of the invention, R¹ is selected from the group comprising or consisting of C₁₋₁₂alkyl-, C₁₋₁₂alkenyl-, C₁₋₁₂alkynyl-, C₁₋₁₂alkoxy-, C₁₋₁₂alkyl-O-C₁₋₁₂alkyl-, C₁₋₁₂alkyl-OC(O)-C₁₋₁₂alkyl-, C₃₋₁₂cycloalkyl-, C₃₋₁₂cycloalkenyl-, C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R¹ may optionally be substituted with one or more substituent selected from the group comprising or consisting of hydrogen, hydroxy, oxy, and C₁₋₆alkoxy-, and wherein R² is selected from the group comprising or consisting of C₁₋₃₀alkyl-, C₁₋₃₀alkenyl-, wherein R³ is selected from the group comprising or consisting of hydrogen, C₁₋₆alkyl-, and wherein R is a bond or C₁₋₆alkyl-.

According to some aspect of the invention, R¹ is selected from the group comprising or consisting of C₁₋₆alkyl-, C₁₋₆alkenyl-, C₁₋₆alkynyl-, C₁₋₆alkoxy-, C₁₋₆alkyl-O-C₁₋₆alkyl-, C₁₋₆alkyl-OC(O)-C₁₋₆alkyl-, C₃₋₆cycloalkyl-, C₃₋₆cycloalkenyl-, C₆aryl-, C₃₋₆cycloalkyl-C₁₋₆alkyl-, C₃₋₆cycloalkenyl-C₁₋₆alkyl- and C₆aryl-C₁₋₃alkyl-, which R¹ may optionally be substituted with one or more substituent selected from the group comprising or consisting of hydrogen, hydroxy, oxy, and C₁₋₆alkoxy-, and wherein R² is selected from the group comprising or consisting of C₁₋₁₈alkyl-, C₁₋₁₈alkenyl-, wherein R³ is selected from the group comprising or consisting of hydrogen, C₁₋₃alkyl-, and wherein R is a bond or C₁₋₃alkyl-.

According to some aspect of the invention, R¹ is selected from the group comprising or consisting of C₁₋₆alkyl-, C₁₋₆alkenyl-, C₁₋₆alkoxy-, C₁₋₆alkyl-O-C₁₋₆alkyl-, C₃₋₆cycloalkyl-, C₃₋₆cycloalkenyl-, C₆₋₇aryl-, C₃₋₆cycloalkyl-C₁₋₃alkyl-, C₃₋₆cycloalkenyl-C₁₋₃alkyl- and C₅₋₇aryl-C₁₋₃alkyl-, which R¹ may optionally be substituted with one or more substituent selected from the group comprising or consisting of hydrogen, hydroxy, oxy, halogen, carboxy, C₁₋₃hydroxyalkyl-, C₁₋₃haloyalkyl-, and C₁₋₃alkoxy-, and wherein R² is selected from the group comprising or consisting of hydrogen, C₅₋₁₈alkyl-, C₅₋₁₈alkenyl-, C₅₋₁₅alkoxy-, C₅₋₁₅alkyl-O-C₁₋₆alkyl-, and C₅₋₁₅alkyl-OC(O)-C₁₋₆alkyl-, which R² may optionally be substituted with one or more substituent selected from the group comprising or consisting of hydrogen, hydroxy, oxy, halogen and carboxy, wherein R³ is selected from the group comprising or consisting of hydrogen, C₁₋₃alkyl-, C₁₋₃alkoxy- and C₁₋₃alkyl-O-C₁₋₃alkyl-, and wherein R is a bond or C₁₋₃alkyl-.

According to some aspect of the invention, R¹ is selected from the group comprising or consisting of hydrogen, C₆₋₇aryl- and C₅₋₇aryl-C₁₋₃alkyl-, which R¹ may optionally be substituted with one or more substituent selected from the group comprising or consisting of hydrogen, hydroxy and C₁₋₃alkoxy-, and wherein R² is selected from the group comprising or consisting of hydrogen, C₅₋₁₅alkyl- and C₅₋₁₅alkenyl-, wherein R³ is selected from the group comprising or consisting of hydrogen, C₁₋₃alkyl- and wherein R is a bond or C₁₋₃alkyl-.

According to some aspect of the invention, R¹ is C₅₋₇aryl-C₁₋₃alkyl-, which R¹ may optionally be substituted with one or more substituent selected from the group comprising hydrogen, hydroxy and C₁₋₃alkoxy-, wherein R² is selected from the group comprising C₅₋₁₆alkyl- and C₅₋₁₅alkenyl-, and wherein R³ is hydrogen, methyl or ethyl, and wherein R is a bond.

The process with these compounds results in improved yields and conversion rates, which is especially important for large scale production.

According to some aspect of the invention, R¹ is C₅₋₇aryl-C₁₋₃alkyl-, which R¹ may optionally be substituted with one or more substituent selected from the group comprising or consisting of hydrogen, hydroxy and C₁₋₃alkoxy-, and wherein R² is selected from the group comprising or consisting of hydrogen, C₅₋₁₆alkyl- and C₅₋₁₅alkenyl-, wherein R³ is hydrogen, methyl or ethyl, and wherein R is a bond or C₁₋₂alkyl-.

According to some aspect of the invention, R¹ is C₆aryl-C₁₋₂alkyl-, which R¹ may optionally be substituted with one or more substituent selected from the group comprising or consisting of hydrogen, hydroxy and C₁₋₂alkoxy-, and wherein R² is selected from the group comprising or consisting of hydrogen, C₇₋₁₀alkyl- and C₇₋₁₀alkenyl-, and wherein R³ is hydrogen, methyl, or ethyl wherein R is a bond or C₁₋₂alkyl-.

According to some aspect of the invention, R¹ is C₆aryl-, or C₆aryl-C₁₋₂alkyl-, optionally be substituted with one or more substituent selected from the group comprising or consisting of hydrogen, hydroxy, oxy, and methoxy-.

According to some aspect of the invention, R² is hydrogen, methanyl, ethanyl, heptanyl, octanyl, 8-methyl-nonanyl, octadecanyl or 8-methyl-nonenyl.

The process with these compounds results in improved yields and conversion rates, which is especially important for large scale production.

One aspect relates to a process for enzymatic synthesis of amides of formula III from amines of formula I and compounds of formula IIa,

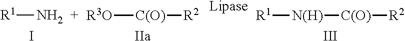

wherein R¹ is selected from the group comprising or consisting of C₁₋₁₂alkyl-, C₁₋₁₂alkenyl-, C₁₋₁₂alkynyl-, C₁₋₁₂alkoxy-, C₁₋₁₂alkyl-O-C₁₋₁₂alkyl-, C₁₋₁₂alkyl-OC(O)-C₁₋₁₂alkyl-, C₁₋₁₂alkyl-NH-C₁₋₁₂alkyl-, C₁₋₁₂alkyl-NHC(O)-C₁₋₁₂alkyl-, C₃₋₁₂cycloalkyl-, C₃₋₁₂cycloalkenyl-, C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R¹ may optionally be substituted with one or more substituent selected from the group comprising or consisting of hydrogen, hydroxy, oxy, halogen, carboxy, amine, amide, C₁₋₆hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S, wherein R² is selected from the group comprising or consisting of hydrogen, C₁₋₃₀alkyl-, C₁₋₃₀alkenyl-, C₁₋₃₀alkynyl-, C₁₋₃₀alkoxy-, C₁₋₃₀alkyl-O-C₁₋₁₂alkyl-, C₁₋₃₀alkyl-OC(O)-C₁₋₁₂alkyl-, C₁₋₃₀alkyl-NH- C₁₋₁₂alkyl-, C₁₋₃₀alkyl-NHC(O)-C₁₋₁₂alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R² may optionally be substituted with one or more substituent selected from the group comprising or consisting of hydrogen, hydroxy, oxy, halogen, carboxy, amine, amide, C₁₋₆hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S, wherein R³ is selected from the group comprising or consisting of hydrogen, C₁₋₆alkyl-, C₁₋₆alkenyl-, C₁₋₆alkynyl-, C₁₋₆alkoxy-, C₁₋₆alkyl-O-C₁₋₆alkyl-, C₁₋₆alkyl-OC(O)-C₁₋₆alkyl-, C₁₋₆alkyl-NH-C₁₋₆alkyl-, C₁₋₆alkyl-NHC(O)-C₁₋₆alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R³ may optionally be substituted with one or more substituent selected from the group comprising or consisting of hydrogen, hydroxy, oxy, halogen, carboxy, amine, amide, C₁₋₆hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S characterized in that the lipase is immobilized on a rotary bed reactor or on a spin-fixed-bed reactor and a Dean-Stark apparatus is used for dehydration.

According to some aspect of the invention, R¹ is selected from the group comprising or consisting of C₁₋₆alkyl-, C₁₋₆alkenyl-, C₁₋₆alkoxy-, C₁₋₆alkyl-O-C₁₋₆alkyl-, C₃₋₆cycloalkyl-, C₃₋₆cycloalkenyl-, C₆₋₇aryl-, C₃₋₆cycloalkyl-C₁₋₃alkyl-, C₃₋₆cycloalkenyl-C₁₋₃alkyl- and C₅₋₇aryl-C₁₋₃alkyl-, which R¹ may optionally be substituted with one or more substituent selected from the group comprising or consisting of hydrogen, hydroxy, oxy, halogen, carboxy, C₁₋₃hydroxyalkyl-, C₁₋₃haloyalkyl-, and C₁₋₃alkoxy-, and wherein R² is selected from the group comprising or consisting of hydrogen, C₅₋₁₅alkyl-, C₅₋₁₅alkenyl-, C₅₋₁₅alkoxy-, C₅₋₁₅alkyl-O-C₁₋₆alkyl-, and C₅₋₁₅alkyl-OC(O)-C₁₋₆alkyl-, which R² may optionally be substituted with one or more substituent selected from the group comprising or consisting of hydrogen, hydroxy, oxy, halogen and carboxy, and wherein R³ is selected from the group comprising or consisting of hydrogen, C₁₋₃alkyl-, C₁₋₃alkoxy- and C₁₋₃alkyl-O-C₁₋₃alkyl-.

According to some aspect of the invention, R¹ is C₅₋₇aryl-C₁₋₃alkyl-, which R¹ may optionally be substituted with one or more substituent selected from the group comprising or consisting of hydrogen, hydroxy and C₁₋₃alkoxy-, and wherein R² is selected from the group comprising or consisting of hydrogen, C₅₋₁₅alkyl- and C₅₋₁₅alkenyl-, and wherein R³ is hydrogen, methyl or ethyl.

According to some aspect of the invention, R¹ is C₆aryl-C₁₋₂alkyl-, which R¹ may optionally be substituted with one or more substituent selected from the group comprising or consisting of hydrogen, hydroxy and C₁₋₂alkoxy-, and wherein R² is selected from the group comprising or consisting of C₇₋₁₀alkyl- and C₇₋₁₀alkenyl-, and wherein R³ is hydrogen, methyl or ethyl.

The process allows for effective and efficient large-scale production of amide compounds like capsaicinoids and derivatives thereof. The process has improved yields compared to known processes. The amidation processes are environmentally friendly and especially cost effective.

According to some aspect of the invention, compounds of formula III are compounds of formula IV

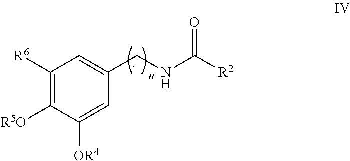

wherein n is 1 or 2, wherein R² is selected from the group comprising or consisting of C₃₋₃₀alkyl-, C₃₋₃₀alkenyl-, C₃₋₃₀alkynyl-, C₃₋₁₂cycloalkyl-, C₃₋₁₂cycloalkenyl- and C₅₋₁₂aryl-, which R² may optionally be substituted with one or more substituent selected from the group comprising or consisting of hydrogen, hydroxy, oxy, halogen, carboxy, amide, C₁₋₆hydroxyalkyl-, C₁₋₆haloyalkyl-, C₁₋₆amideyalkyl-, C₁₋₆carboxyalkyl-, C₁₋₆sulfuralkyl-, C₁₋₆sulfidealkyl- and C₁₋₆alkoxy- and C₅₋₁₂aryl-, and wherein one or more carbon in a cycloalkyl, cycloalkenyl or aryl may be substituted with one or more heteroatoms selected from O, N or S, wherein R⁴ or R⁵ is independently selected from the group comprising or consisting of hydrogen, C₁₋₆alkyl-, C₂₋₆alkenyl-, C₂₋₆alkynyl-, C₃₋₁₀cycloalkyl-, C₃₋₁₀cycloalkenyl- and C₅₋₁₂aryl-, which R⁴ or R⁵ may optionally be independently substituted with one or more substituent selected from the group comprising or consisting of hydroxy, oxy, halogen, carboxy, amine, amide, C₁₋₆hydroxyalkyl-, C₁₋₆haloyalkyl-, C₁₋₆amineoxyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S, wherein R⁶ is selected from the group comprising or consisting of hydrogen, hydroxy, oxy, halogen, carboxy, amine, amide, C₁₋₁₀alkyl-, C₂₋₁₀alkenyl-, C₂₋₁₀alkynyl-, C₃₋₁₂cycloalkyl-, C₃₋₁₂cycloalkenyl- and C₅₋₁₂aryl-, which R⁶ may optionally be substituted with one or more substituent selected from the group comprising or consisting of hydrogen, hydroxy, oxy, halogen, carboxy, amine, amide, C₁₋₆hydroxyalkyl-, C₁₋₆haloyalkyl-, C₁₋₆amineoxyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S.

The process with these compounds results in improved yields and conversion rates, which is especially important for large scale production.

In some aspects, wherein compounds of formula III are compounds of formula IV, n is 1 or 2, R² is selected from the group comprising or consisting of C₃₋₃₀alkyl-, C₃₋₃₀alkenyl-, R⁴ or R⁵ is independently selected from the group comprising or consisting of hydrogen, C₁₋₃alkyl, and R⁶ is hydrogen.

In some aspects, wherein compounds of formula III are compounds of formula IV, n is 1 or 2, R² is selected from the group comprising C₃₋₁₈alkyl- and C₃₋₁₈alkenyl-, R⁴ or R⁵ is independently selected from the group comprising hydrogen, C₁₋₆alkyl-, and R⁶ is hydrogen.

In some aspects, wherein compounds of formula III are compounds of formula IV n is 1 or 2, R² is selected from the group comprising C₅₋₁₆alkyl- and C₅₋₁₅alkenyl-, R⁴ or R⁵ is independently selected from the group comprising hydrogen, C₁₋₃alkyl-, and R⁶ is hydrogen.

In some aspects, wherein compounds of formula III are compounds of formula IV, R² is methanyl, ethanyl, heptanyl, octanyl, 8-methyl-nonanyl or octadecanyl or 8-methyl-nonenyl.

The process with these compounds results in improved yields and conversion rates, which is especially important for large scale production.

According to some aspect of the invention, no solvent is used.

According to some aspect of the invention, the solvent is an organic solvent selected from the group comprising or consisting of methyl tert-butyl ether, diisopropylether, C₁₋₆alkyl-O-C₁₋₆alkyl ethers, hexane and other C₅₋₁₀alkanes, cyclohexane and other C₅₋₁₀cycloalkanes, benzene, toluene, xylene, tert-butanol, tert amyl alcohol, other bulky secondary or tertiary C₅₋₁₀ alcohols and any esters thereof. In some aspects, the organic solvent is selected from the group comprising or consisting of diisopropylether, cyclohexane, toluene and tert-butanol, or mixtures thereof. In some aspects, the solvent is cyclohexane, toluene or diisopropylether (DIPE). In some aspects, the solvent is diisopropylether (DIPE). In some aspects, the solvent is cyclohexane. In some aspects, the solvent is toluene. In some aspects, the solvent is tert-butanol.

According to some aspect of the invention, when R³ is not hydrogen, the solvent is selected from the group comprising or consisting of methyl tert-butyl ether, diisopropylether, C₁₋₆alkyl-O-C₁₋₆alkyl ethers, hexane and other C₅₋₁₀alkanes, cyclohexane and other C₅₋₁₀cycloalkanes, benzene, toluene, xylene, tert-butanol, tert amyl alcohol, other bulky secondary or tertiary C₅₋₁₀ alcohols and their esters. In some aspects, the organic solvent is selected from the group comprising or consisting of diisopropylether, cyclohexane, toluene and tert-butanol, or mixtures thereof. In some aspects, the solvent is cyclohexane, toluene or diisopropylether (DIPE). In some aspects, the solvent is diisopropylether (DIPE). In some aspects, the solvent is cyclohexane. In some aspects, the solvent is toluene. In some aspects, the solvent is tert-butanol. In some aspects, the solvent is recyclable. In some aspects, the solvent is recycled. In some aspects, the solvent is recycled for at least 70% or 80% or 90%. Recycling the solvent reduces the overall costs for the process and also reduced the carbon footprint of the process.

According to some aspect of the invention, the lipase is selected from the group comprising or consisting of Candida antarctica lipase A, Candida antarctica lipase B, cross-linked Substilisin A protease, Porcine pancreas lipase, Candida cylindracea lipase, Rhizopus arrhizus, Penicillum cyclopium, Mucor miehei, Thermomyces lanuginosus lipase, Candida rugosa lipase and Pseudomonas lipoprotein lipase. In one aspect, the lipase is selected from the group comprising or consisting of Candida antarctica lipase A and Candida antarctica lipase B. In one aspect, the lipase is Candida antarctica lipase. In one aspect, the lipase is Candida antarctica lipase B (Novozym 435™). The immobilized enzymes, like Candida antarctica Lipase B or C. antarctica lipase A, are commercially and easily available under tradenames like Novozym 435™. The availability at relative low cost is important for a cost-effective process, especially for large scale processes.

According to some aspect of the invention, the process temperature is between 15° C. and 150° C., or between 15° C. and 115° C., or between 50 and 90, or 70 -80° C. The relative low temperature is important for a cost-effective process, especially for large scale processes.

According to some aspect of the invention, the process is performed at a pressure between 0.090 and 0.200 MPa, or at atmospheric pressure (about 0.1 MPa). Performing the process at atmospheric pressure is important for a cost-effective process, especially for large scale processes.

According to some aspect of the invention, the rotary bed reactor is loaded for 10 to 75 wt % with the lipase. According to some aspect of the invention, the rotary bed reactor is loaded for 11 to 60 wt % with the lipase. According to some aspect of the invention, the rotary bed reactor is loaded for 15 to 50 wt % with the lipase. The unique combination of an immobilized enzyme on a rotary bed reactor or on a spin-fixed-bed reactor and a Dean Trap apparatus improves the conversion rate and yield of the process. Because the process is both time- and cost-effective, a possible additional cost for loading of the lipase with more than 10 wt % loading becomes affordable.

According to some aspect of the invention, the rate of agitation is 150 to 600 rpm or 200 to 500 rpm, or 200 to 450 rpm.

The invention also relates to a process for synthesis of compounds of formula II, wherein R² is a C₆₋₁₈alkyl or C₆₋₁₈alkenyl. According to some aspect, compounds of formula II, wherein R² is a C₆₋₁₈alkyl or C₆₋₁₈alkenyl, which may be straight or branched, are prepared comprising the steps of

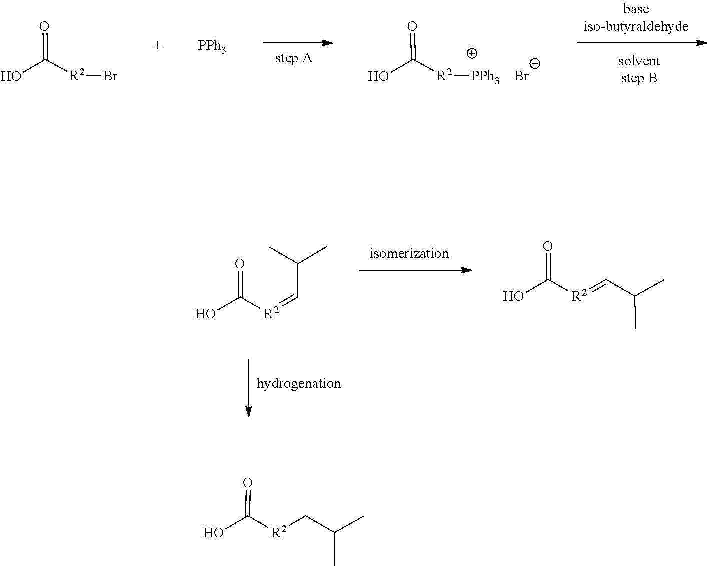

step A-1, wherein the reaction is performed without solvent or with an organic solvent, step B-1, wherein a solvent is an aprotic organic solvent, and step B-1, wherein a base is a sodium or potassium alkoxides, optionally isomerization step C-1, wherein a catalyst is selected from the group comprising or consisting of HNO₂, HNO₃ and combinations of NaNO₂/HNO₃, NaNO₂/NaNO₃/H₂SO₄, that can generate HNO₂ or HNO₃, and hydrogenation step D-1, wherein a catalyst is a heterogeneous hydrogenation catalyst and a hydrogen source is hydrogen gas.

In some aspects, R² is a C₆₋₁₀alkyl.

In some aspects, the organic solvent in step A-1 is ethyl acetate, wherein the aprotic organic solvent in step B-1 is selected from the group comprising or consisting of 2-methyl tetrahydrofuran, tetrahydrofuran and toluene, wherein the sodium or potassium alkoxide base in step B-1 is selected from the group comprising or consisting of NaH, KH, t-BuOK, t-BuONa, and wherein the heterogeneous hydrogenation catalyst in hydrogenation step D-1 is selected from the group comprising or consisting of Pd/C and Pd/Al₂O₃.

In some aspects, the organic solvent in step A-1 is ethyl acetate, the aprotic organic solvent in step B-1 is 2-methyl tetrahydrofuran, the sodium or potassium alkoxide base in step B-1 is t-BuOK, and the heterogeneous hydrogenation catalyst in hydrogenation step D-1 is Pd/C.

In the production of 8-methyl-6-nonenoic acid, using 2-MeTHF as recyclable solvent for the key Wittig reaction between (6-Carboxyhexyl)triphenylphosphonium bromide and iso-butyraldehyde improves conversion rate and yield. The synthesis is time- and cost-effective with high yields and conversion rates. This is especially important for large scale process.

Further solvents may be used in the process steps. Extraction and filtration may be performed between the steps.

The process may be performed at room temperature. The process can be performed at atmospheric pressure (approximately 1 atm or 0.1 MPa).

The invention also relates to a process for a new synthetic route to 8-methyl-6-nonanoic acid, which is used for the direct production of dihydro-capsaicin. The process starts from cyclohexanone and iso-butyraldehyde as raw materials, with aldol condensation, Baeyer-Villiger oxidation and hydrogenation as key steps.

According to some aspect of the invention, compounds of formula II, wherein R² is 8-methyl-nonanyl, are prepared comprising the steps of

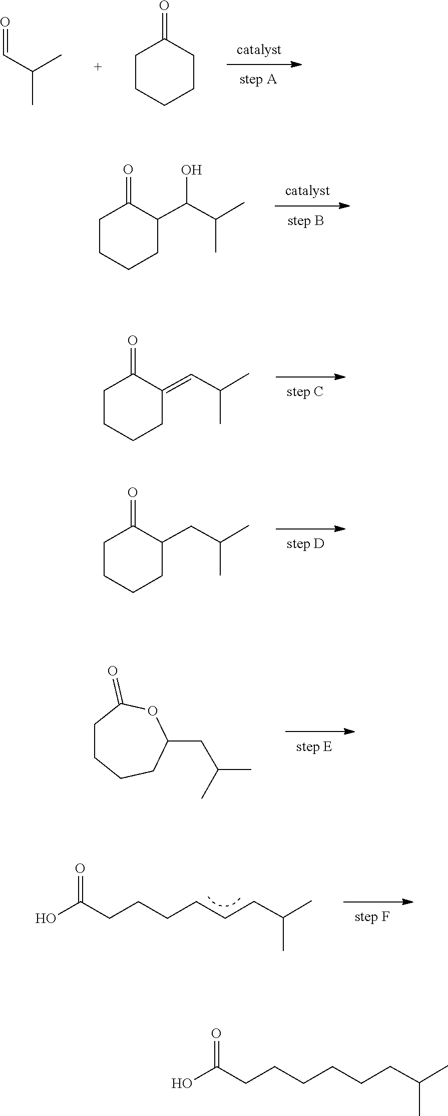

step A-2, wherein the reaction is performed without solvent or with any organic solvent and a catalyst is selected from the group comprising or consisting of amines and inorganic bases, step B-2, wherein the reaction is performed without solvent or with an organic solvent, and a catalyst is an acid, step C-2, wherein a catalyst is a heterogeneous hydrogenation catalyst, and a hydrogen source is hydrogen gas, step D-2, wherein an oxidant is a peroxide, and a catalyst is a lipase, and step E-2, wherein a reaction medium is an acidic media, and step F-2, wherein a catalyst is a heterogeneous hydrogenation catalyst, and a hydrogen source is hydrogen gas.

According to some aspects, the organic solvent in step A-2 is selected from the group comprising or consisting of toluene and aromatic solvents, THF and ethers, dichloromethane and halogenated solvents, and the catalyst is selected from the group comprising or consisting of pyrrolidine and corresponding salts, NaOH and KOH, wherein the organic solvent in step B-2 is selected from the group comprising or consisting of toluene, and the acid is selected from the group comprising or consisting of p-TsOH, sulfuric acid and Amberlyst-15, wherein the catalyst in step C-2 is selected from the group comprising or consisting of Pd/C, Pd/Al₂O₃, wherein the oxidant in step D-2 is selected from the group comprising or consisting of aqueous H₂O₂ and peroxy acids and the lipase is selected from the group comprising or consisting of Candida antarctica lipase A, Candida antarctica lipase B, cross-linked Substilisin A protease, Porcine pancreas lipase, Candida cylindracea lipase, Rhizopus arrhizus, Penicillum cyclopium, Mucor miehei, Thermomyces lanuginosus lipase, Candida rugosa lipase and Pseudomonas lipoprotein lipase, and wherein the reaction medium in step E-2 is selected from the group comprising or consisting of aqueous sulfuric acid solution, and wherein the catalyst in step F-2 is selected from the group comprising or consisting of Pd/C, Pd/Al₂O₃, Pd/molecular sieves, Pt/C, Pt/Al₂O₃, and Pt/molecular sieves.

A Dean Stark trap may be used in step B-2.

According to some aspects, the organic solvent in step A-2 is toluene, and the catalyst is pyrrolidine, the organic solvent in step B-2 is toluene and the acid is p-TsOH, the catalyst in step C-2 is Pd/C, the oxidant in step D-2 is aqueous H₂O₂ and the lipase is Candida antarctica lipase B, the reaction medium in step E-2 is aqueous sulfuric acid solution, and the catalyst in step F-2 is Pd/C.

The synthesis is time and cost effective with high yields and conversion rates. This is especially important for large scale process.

Further solvents may be used in the process steps. Extraction and filtration may be performed between the steps.

The process may be performed at room temperature. The process can be performed at atmospheric pressure (approximately 1 atm or 0.1 MPa).

The process as defined anywhere herein are useful for large scale production of compounds of formula III. In some aspects, the process is used for large scale production (>0.5 or >1 kg) of compounds of formula III.

### BRIEF DESCRIPTION OF THE DRAWINGS

The invention will now be explained more closely by the description of different embodiments of the invention and with reference to the appended figures.

FIG. 1 shows a system for performing the process of the invention.

### DETAILED DESCRIPTION OF VARIOUS EMBODIMENTS OF THE INVENTION

### Definitions

Room temperature is a temperature between 15 and 25° C.

EtOAc is ethyl acetate.

DIPE is diisopropylether.

KOtBu is potassium tert-butoxide.

2-MeTHF is 2-methyltetrahydrofuran.

ET2O is diethyl ether.

AcOH is acetic acid.

p-TsOH is p-toluenesulfonic acid or tosylic acid.

tBuOH is tert-butyl alcohol.

equiv. is equivalent. equivalent

As used herein, the term “wt %” or “w/w %” or “w %” means weight percentage, which is a percentage of the total weight.

As used herein, the term “optional” or “optionally” means that the subsequently described event or circumstance may but need not occur, and that the description includes instances where the event or circumstance occurs and instances where it does not.

As used herein, the terms “Cₙ”, used alone or as a suffix or prefix, is intended to include hydrocarbon-containing groups; n is an integer from 1 to 30.

As used herein, the term “halogen” or “halo”, used alone or as suffix or prefix, is intended to include bromine, chlorine, fluorine, and iodine.

As used herein, the term “hetero”, used alone or as a suffix or prefix, is intended to include alkyl, cycloalkyl and aryl groups in which one or more of the carbon atoms (and certain associated hydrogen atoms) are independently replaced with the same or different hetero atoms (S, O or N) or heteroatomic groups. Examples of heteroatomic groups include, but are not limited to, —O—, —S—, —O—O—, —S—S—, —O—S—, NR, ═N—N═, —N═N—, —N═N—NR—, —PR—, —P(O)₂—, —POR—, —O—P(O)₂—, —SO—, —SO₂—, —Sn(R)₂—, and the like.

As used herein, the term “C₁₋₃₀-alkyl”, used alone or as a suffix or prefix, is intended to include both branched and straight chain saturated aliphatic hydrocarbon groups having from 1 to 30 carbon atoms. Examples of C₁₋₄-alkyl include methyl, ethyl, n-propyl, i-propyl, n-butyl, i-butyl, sec-butyl, and tert-butyl.

The term “alkenyl” refers to a monovalent straight or branched chain hydrocarbon radical having at least one carbon-carbon double bond and comprising at least 2 up to about 30 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “alkynyl” refers to a monovalent straight or branched chain hydrocarbon radical having at least one carbon-carbon triple bond and comprising at least 2 and up to about 12 carbon atoms. The triple bond of an alkynyl can be unconjugated or conjugated to another unsaturated group. Suitable alkynyl groups include, but are not limited to C₂₋₆alkynyl groups, such as acetylenyl, methylacetylenyl, butynyl, pentynyl, hexynyl. An alkynyl can be unsubstituted or substituted with one or two suitable substituents.

As used herein, the term “C₁₋₆-alkoxy”, used alone or as a suffix och prefix, refers to a C₁₋₆-alkyl radical, which is attached to the remainder of the molecule through an oxygen atom. Examples of C₁₋₄-alkoxy include methoxy, ethoxy, n-propoxy, i-propoxy, n-butoxy, i-butoxy, sec-butoxy and tert-butoxy.

As used herein, the term “cycloalkyl”, and “cycloalkenyl” used alone or as a suffix or prefix, is intended to include saturated or partially unsaturated cyclic alkyl radical. Where a specific level of saturation is intended, the nomenclature cycloakanyl or cycloalkenyl is used. Examples of cycloalkyl groups include, but is not limited to, groups derived from cyclopropane, cyclobutene, cyclopentane, cyclohexane and the like.

As used herein, the term “aryl” refers to either a monocyclic aromatic ring having 5 or 12 ring members or a multiple ring system having at least one carbocyclic aromatic ring fused to at least one carbocyclic aromatic ring, cycloalkyl ring or a heterocycloalkyl ring. For example, aryl includes a phenyl ring fused to a 5- to 7-membered heterocycloalkyl ring containing one or more heteroatoms independently selected from N, O, and S.

As used herein, the term “C₅₋₁₂-aryl-C₁₋₆-alkyl” refers to a phenyl group that is attached through a C₁₋₆-alkyl radical. Examples of C₆-aryl-C₁₋₃-alkyl include phenylmethyl (benzyl), 1-phenylethyl and 2-phenylethyl.

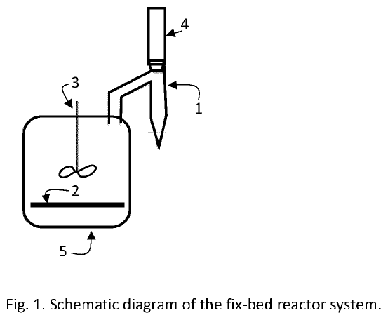

FIG. 1 shows a system for performing the process. In a reactor 5, the lipase is immobilized on a rotary fix bed 2. A motor 3 is used for rotation of the fixed bed 2. The reactor 5 is connected to a Dean Stark apparatus 1, which is connected to a condenser 4.

In the present invention, the process is performed using the lipase, which is immobilized on a rotary bed reactor together with a Dean-Stark apparatus for dehydration.

This process may be used for the preparation of capsaicinoids, but also for the amidation of numerous of other amines with carboxylic acids or esters.

The process may be used for synthesis of amides of formula III from amines of formula I and compounds of formula II or IIa as shown below,

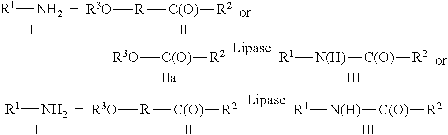

R¹ may be selected from the group comprising C₁₋₁₂alkyl-, C₁₋₁₂alkenyl-, C₁₋₁₂alkynyl-, C₁₋₁₂alkoxy-, C₁₋₁₂alkyl-O-C₁₋₁₂alkyl-, C₁₋₁₂alkyl-OC(O)-C₁₋₁₂alkyl-, C₁₋₁₂alkyl-NH-C₁₋₁₂alkyl-, C₁₋₁₂alkyl-NHC(O)-C₁₋₁₂alkyl-, C₃₋₁₂cycloalkyl-, C₃₋₁₂cycloalkenyl-, C₅₋₁₂aryl-, C₃₋₁₂cycloalkyl-C₁₋₆alkyl-, C₃₋₁₂cycloalkenyl-C₁₋₆alkyl- and C₃₋₁₂aryl-C₁₋₆alkyl-, which R¹ may optionally be substituted with one or more substituent selected from the group comprising hydrogen, hydroxy, oxy, halogen, carboxy, amine, amide, C₁₋₁₅hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-.

R¹ may be selected from the group comprising C₁₋₆alkyl-, C₁₋₆alkenyl-, C₁₋₆alkoxy-, C₁₋₆alkyl-O-C₁₋₆alkyl-, C₃₋₆cycloalkyl-, C₃₋₆cycloalkenyl-, C₆₋₇aryl-, C₃₋₆cycloalkyl-C₁₋₃alkyl-, C₃₋₆cycloalkenyl-C₁₋₃alkyl- and C₅₋₇aryl-C₁₋₃alkyl-, which R¹ may optionally be substituted with one or more substituent selected from the group comprising hydrogen, hydroxy, oxy, halogen, carboxy, C₁₋₃hydroxyalkyl-, C₁₋₃haloyalkyl-, and C₁₋₃alkoxy-.

Or R¹ may be C₅₋₇aryl-C₁₋₃alkyl-, or C₆₋₇aryl-C₁₋₂alkyl-, or C₆aryl-C₁₋₃alkyl-, optionally substituted with hydrogen, hydroxy and/or methoxy.

R² may be selected from the group comprising hydrogen, C₁₋₃₀alkyl-, C₁₋₃₀alkenyl-, C₁₋₃₀alkynyl-, C₁₋₃₀alkoxy-, C₁₋₃₀alkyl-O-C₁₋₁₂alkyl-, C₁₋₃₀alkyl-OC(O)-C₁₋₁₂alkyl-, C₁₋₃₀alkyl-NH-C₁₋₁₂alkyl-, C₁₋₃₀alkyl-NHC(O)-C₁₋₁₂alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and C₅₋₁₂aryl-C₁₋₆alkyl-, which R² may optionally be substituted with one or more substituent selected from the group comprising hydrogen, hydroxy, oxy, halogen, carboxy, amine, amide, C₁₋₆hydroxyalkyl-, C₁₋₁₅haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-.

R² may be selected from the group comprising hydrogen, C₃₋₃₀alkyl-, C₃₋₃₀alkenyl-, C₃₋₃₀alkynyl-, C₃₋₁₂cycloalkyl-, C₃₋₁₂cycloalkenyl- and C₅₋₁₂aryl-.

R² may be selected from the group comprising hydrogen, C₅₋₁₈alkyl-, C₅₋₁₈alkenyl-, C₅₋₁₈alkoxy-, C₅₋₁₈alkyl-O-C₁₋₆alkyl-, and C₅₋₁₈alkyl-OC(O)-C₁₋₆alkyl-, which R² may optionally be substituted with one or more substituent selected from the group comprising hydrogen, hydroxy, oxy, halogen and carboxy.

Or R² may be selected from the group comprising hydrogen, C₅₋₁₆alkyl- and C₅₋₁₆alkenyl- or C₇₋₁₇alkyl- and C₇₋₁₆alkenyl-, or C₇₋₁₀alkyl- and C₇₋₁₀alkenyl-.

R³ is selected from the group comprising hydrogen, C₁₋₆alkyl-, C₁₋₆alkenyl-, C₁₋₆alkynyl-, C₁₋₆alkoxy-, C₁₋₆alkyl-O-C₁₋₆alkyl-, C₁₋₆alkyl-OC(O)-C₁₋₆alkyl-, C₁₋₆alkyl-NH-C₁₋₆alkyl-, C₁₋₆alkyl-NHC(O)-C₁₋₆alkyl-, C₃₋₁₂cycloalkyl-, C₃₋₁₂cycloalkenyl- and C₅₋₁₂aryl-, C₃₋₁₂cycloalkyl-C₁₋₆alkyl-, C₃₋₁₂cycloalkenyl-C₁₋₆alkyl- and Cs-12aryl-C₁₋₆alkyl-, which R³ may optionally be substituted with one or more substituent selected from the group comprising hydrogen, hydroxy, oxy, halogen, carboxy, amine, amide, C₁₋₆hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-, and wherein one or more carbon in a cycloalkyl, cycloalkenyl or aryl may be substituted with one or more heteroatoms selected from O, N or S.

R³ may be selected from the group comprising hydrogen, C₁₋₃alkyl-, C₁₋₃alkoxy- and C₁₋₃alkyl-O-C₁₋₃alkyl-.

Or R³ may be hydrogen, methyl, ethyl. R³ may be hydrogen.

R may be a bond. R may be C₁₋₃alkyl-, or methyl or ethyl.

The compounds of formula III may be represented the structure of IV

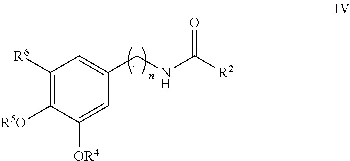

wherein n is 1 or 2, wherein R² is selected from the group comprising C₃₋₂₀alkyl-, C₃₋₂₀alkenyl-, C₃₋₂₀alkynyl-, C₃₋₁₂cycloalkyl-, C₃₋₁₂cycloalkenyl- and C₅₋₁₂aryl-, which R² may optionally be substituted with one or more substituent selected from the group comprising hydrogen, hydroxy, oxy, halogen, carboxy, amine, amide, C₁₋₆hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy- and C₅₋₁₂aryl-, wherein R⁴ or R⁵ is selected from the group comprising hydrogen, C₁₋₆alkyl-, C₂₋₆alkenyl-, C₂₋₆alkynyl-, C₃₋₁₀cycloalkyl-, C₃₋₁₀cycloalkenyl- and C₅₋₁₂aryl-, wherein R⁶ is selected from the group comprising hydrogen, hydroxy, oxy, halogen, carboxy, amine, amide, C₁₋₆alkyl-, C₂₋₆alkenyl-, C₂₋₆alkynyl-, C₃₋₆cycloalkyl-, C₃₋₆cycloalkenyl- and C₅₋₆aryl-, which R⁶ may optionally be substituted with one or more substituent selected from the group comprising hydrogen, hydroxy, oxy, halogen, carboxy, amine, amide, C₁₋₆hydroxyalkyl-, C₁₋₆haloyalkyl-, C₁₋₆aminexyalkyl-, C₁₋₆amideyalkyl-, C₁₋₆carboxyalkyl-, C₁₋₆sulfuralkyl-, C₁₋₆sulfidealkyl- and C₁₋₆alkoxy-, and.

The compounds of formula III may be represented the structure of IV wherein n is 1 or 2, wherein R² is selected from the group comprising or consisting of C₅₋₁₈alkyl-, C₅₋₁₈alkenyl-, Cs-15alkoxy-, C₅₋₁₈alkyl-O-C₁₋₆alkyl-, and C₅₋₁₈alkyl-OC(O)-C₁₋₆alkyl-, which R² may optionally be substituted with one or more substituent selected from the group comprising or consisting of hydroxy, oxy, halogen and carboxy, wherein R⁴ or R⁵ is selected from the group comprising or consisting of hydrogen and C₁₋₃alkyl-, and R⁶ may be selected from the group comprising or consisting of hydrogen, hydroxy and oxy.

The compounds of formula III may be represented the structure of IV wherein n is 1 or 2, wherein R² is selected from the group comprising C₆₋₁₂alkyl- and C₆₋₁₂alkenyl-, or C₇₋₁₀alkyl- and C₇₋₁₀alkenyl-, wherein R⁴ or R⁵ is selected from the group comprising hydrogen, methyl or ethyl, and R⁶ is hydrogen.

### Prior Art Processes for Preparation of Capsaicinoids

Ester as acyl donor: need very dry amine (<3wt % water content),

otherwise water will cover the amine on the bottom and retard the reaction. It is difficult to reach full conversion of the ester without addition of excess amine.

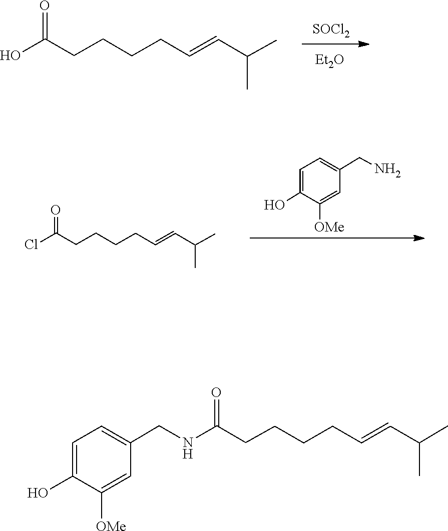

Expensive anhydrous solvent and toxic SOCl₂ were required in this process. Comparing with the enzymatic process, the yield was much lower and the resulting appearance of the product was much worse. The product was sticky with brownish-yellow color. See example 25.

### Enzymatic Process

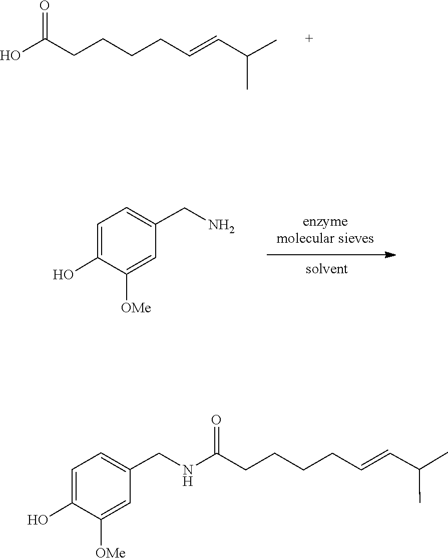 [SMILES: CC(C)/C=C/CCCCC(=O)O ; COc1cc(CN)ccc1O ; COc1cc(CNC(=O)CCCC/C=C/C(C)C)ccc1O]

This process requires large amounts of molecular sieves, which require bigger equipment when scaling up. Filtration and purification is necessary, which is time- and cost-consuming. See example 23.

Both prior art processes are time consuming, expensive with yields that are too low to be used for large scale production in an economically feasible manner.

The process of the invention may be used for the preparation of capsaicinoids according to the scheme below.

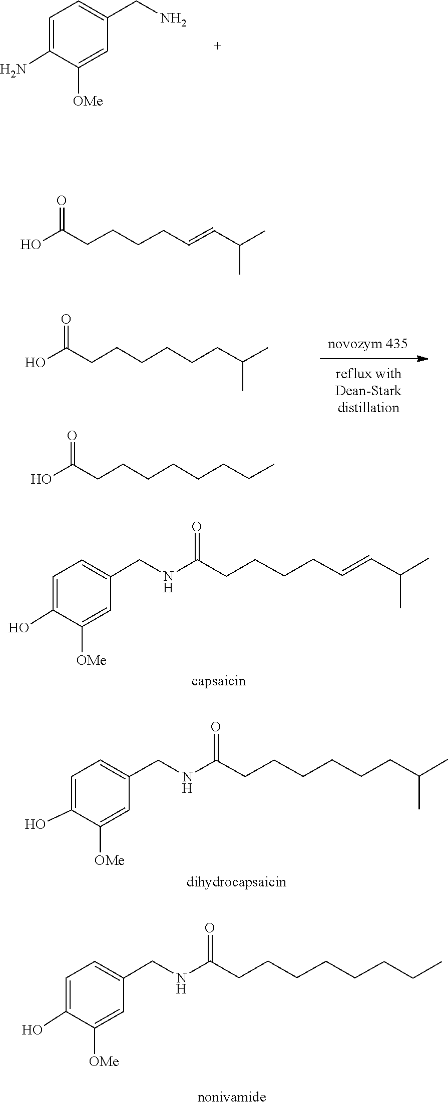

In the process of the invention, no solvent may be used.

If a solvent is used, the solvent may be an organic solvent selected from the group comprising or consisting of methyl tert-butyl ether, diisopropylether, C₁₋₆alkyl-O-C₁₋₆alkyl ethers, hexane and other C₅₋₁₀alkanes, cyclohexane and other C₅₋₁₀cycloalkanes, benzene, toluene, xylene, tert-butanol, tert amyl alcohol, other bulky secondary or tertiary C₅₋₁₀ alcohols and any esters thereof. The solvent may be toluene, diisopropylether or cyclohexane.

When R³ is not hydrogen, the solvent may be selected from the group comprising or consisting of methyl tert-butyl ether, diisopropylether, C₁₋₆alkyl-O-C₁₋₆alkyl ethers, hexane and other C₅₋₁₀alkanes, cyclohexane and other C₅₋₁₀cycloalkanes, benzene, toluene, xylene, tert-butanol, tert amyl alcohol, other bulky secondary or tertiary C₅₋₁₀ alcohols and their esters.

The solvent may be toluene, diisopropylether or cyclohexane.

The lipase may be selected from the group comprising or consisting of Candida antarctica lipase A, Candida antarctica lipase B, cross-linked Substilisin A protease, Porcine pancreas lipase, Candida cylindracea lipase, Rhizopus arrhizus, Penicillum cyclopium, Mucor miehei, Thermomyces lanuginosus lipase, Candida rugosa lipase and Pseudomonas lipoprotein lipase.

The process may be performed at a temperature between room temperature and 150° C., or between room temperature and 115° C.

The process may be performed at a pressure between 0.090 and 0.200 MPa, or about 0.1 MPa.

The compounds of formula Il may be prepared comprising or consisting of the steps of

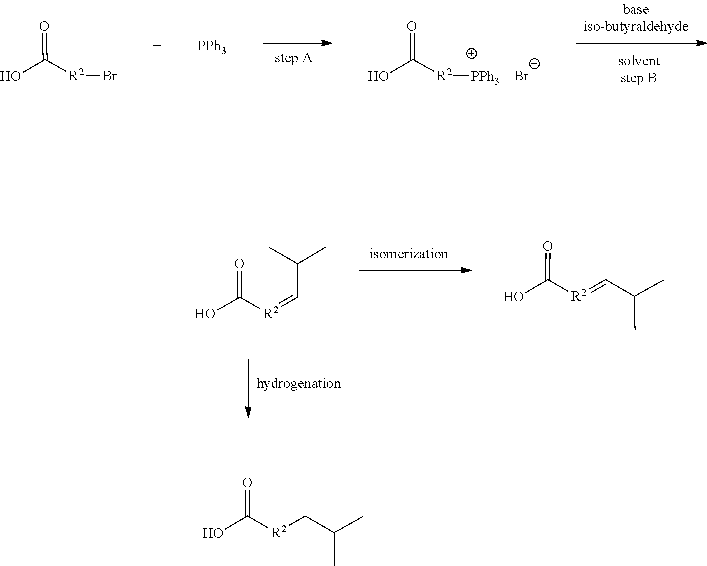

wherein R² is a C₆₋₁₈alkyl or C₆₋₁₈alkenyl, which may be straight or branched, step A-1, the reaction is performed without solvent or with any organic solvent, such as EtOAc, step B-1, a solvent is selected from the group comprising or consisting of 2-methyl tetrahydrofuran, tetrahydrofuran, toluene and any other aprotic organic solvent, step B-1, a base is selected from the group comprising or consisting of NaH, KH, t-BuOK, t-BuONa and another sodium or potassium alkoxides, isomerization step C-1, a catalyst is selected from the group comprising or consisting of HNO₂, HNO₃ and any other combination that can generate HNO₂ or HNO₃, and hydrogenation step D-1, a catalyst is selected from the group comprising or consisting of Pd/C, Pd/Al₂O₃ and any other heterogeneous hydrogenation catalyst, a hydrogen source is hydrogen gas.

The compounds of formula II may be prepared comprising or consisting of the steps of

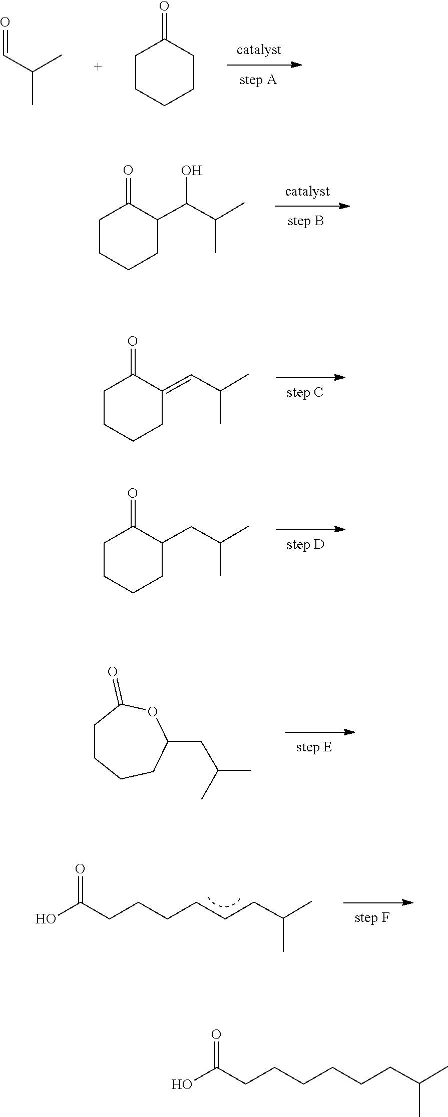

step A-2, the reaction is performed without solvent or with any organic solvents, such as toluene, a catalyst is selected from the group comprising or consisting of pyrrolidine, other amines and corresponding salts, NaOH, KOH, and other inorganic bases, step B-2, the reaction is performed without solvent or with an organic solvent, such as toluene, a catalyst is selected from the group c comprising or consisting of p-TsOH, sulfuric acid, Amberlyst-15, and other acids, step C-2 and step F-2, a hydrogen source is hydrogen gas, a catalyst is selected from the group comprising or consisting of Pd/C, Pd/Al₂O₃ and another heterogeneous hydrogenation catalyst, step D-2, an oxidants is selected from the group comprising or consisting of aqueous H₂O₂, peroxyacids and another peroxides, a catalyst is selected from the group comprising or consisting of Candida antarctica lipase A, Candida antarctica lipase B, cross-linked Substilisin A protease, Porcine pancreas lipase, Candida cylindracea lipase, Rhizopus arrhizus, Penicillum cyclopium, Mucor miehei, Thermomyces lanuginosus lipase, Candida rugosa lipase and Pseudomonas lipoprotein lipase, and step E-2, a reaction medium is selected from the group comprising or consisting of aqueous sulfuric acid solution or another strong acidic media, and Step F-2, a catalyst is selected from the group comprising or consisting of Pd/C, Pd/Al₂O₃, Pd/ molecular sieves, Pt/C, Pt/Al₂O₃, and Pt/molecular sieves, a hydrogen source is hydrogen gas.

The processes for the preparation of compounds of formula II may be performed at a temperature is between room temperature and 150° C., or between room temperature and 115 ° C. These processes may be performed at a pressure between 0.090 and 0.200 MPa, or about 0.1 MPa.

### EXPERIMENTAL SECTIONS

### Preparation of Vanillylamine

Vanillylamine was prepared from its hydrochloride salt. The HCl salt was purchased from commercial suppliers or prepared according to literature procedures (ChemBioChem 2009, 10, 823; J. Med. Chem. 2018, 61, 8225.).

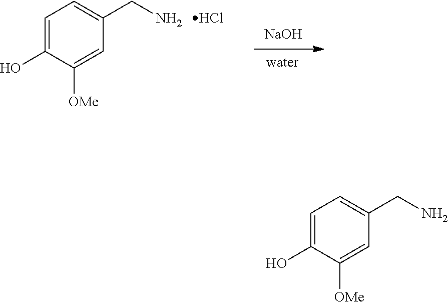 [SMILES: COc1cc(CN)ccc1O ; Cl ; "NaOH" ; COc1cc(CN)ccc1O]

### Example 1

50.00 g of vanillylamine HCl salt was dissolved in 500 mL of cold water (˜5° C.), and cooled with an ice-bath, 1 equivalent of 3 M NaOH (87.9 mL) was portion-wise added in 10 min while keeping vigorous stirring. The internal temperature kept about 5° C. After addition of all bases, the milky solution was stirred for further 5 min, then filtered. The white product in the funnel was washed twice with cold water (5° C., 100 mL×2), then dried under vacuum until the weight remains the same. 37.32 g (92.4% yield) of product was obtained.

### Example 2

500.0 g of vanillylamine HCl salt was dissolved in 5 L of water (10˜15° C.), 1 equivalent of 3 M NaOH was portion-wise added in 20 min while keeping vigorous stirring. After addition of all bases, the milky solution was stirred for further 10 min, then filtered. The white product in the funnel was washed twice with cold water (1 L×2), then dried in a vacuum chamber at 50° C. for 24 hours. 478.0 g of off-white product was obtained with 19.5 wt % moisture content (determined with Kern DBS 60-3 moisture analyser).

### Preparation of Fatty Acids

Preparation of Fatty Acids with Wittig Reaction as Key Step

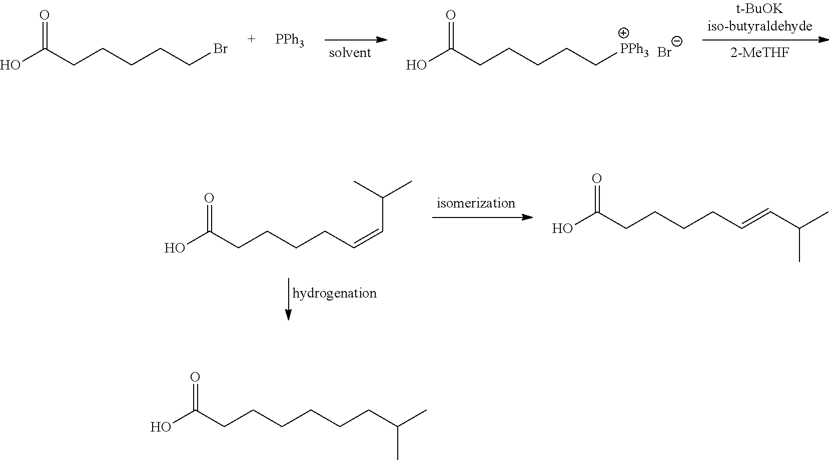

### Example 3

### Preparation of (6-Carboxyhexyl)triphenylphosphonium Bromide

In a 1 L round-bottom flask, 97.53 g 6-bromohexanoic acid and 131.15 g (1.0 equiv) triphenylphosphine (PPh₃) were dissolved in 500 mL EtOAc. The mixture was heated at 75-80° C. and stirred for 7 days. After filtration, the collected product was washed with EtOAc (50 mL×2) and then dried under vacuum to give 221.80 g white powder (97.0% yield). The combined filtrate was recycled as solvent for more batches.

### Example 4

### Preparation of (Z)-8-methyl-6-nonenoic Acid

In a 1 L two-necked round-bottom flask 100.00 g (6-Carboxyhexyl)triphenylphosphonium (Ph₃PO) bromide and 49.07 g (2.0 equiv) KOtBu were dissolved in 300 mL 2-MeTHF under protection of nitrogen atmosphere and cooled using an ice water bath. The reaction mixture turned bright orange color while the compounds were dissolved. A solution of 18.92 g (1.2 equiv) isobutyraldehyde in 200 mL 2-MeTHF was slowly added to the cold reaction mixture, which quickly turned white. After the addition was completed, the reaction mixture was warmed to room temperature and stirred for 6 h. The reaction was quenched by addition of 500 mL H₂O. The MeTHF solvent was recovered by distillation. After cooling to room temperature, most of Ph₃PO was precipitated and collected as white powder by filtration. The filtrate was acidified with concentrated HCl to pH 2, the formed organic layer was collected, and the water phase was extracted with Et₂O (100 mL×2). The organic phases were combined, dried with anhydrous Na₂SO₄, and concentrated to give 56.6 g crude product. This crude product was then distilled under reduced pressure to give (Z)-8-methyl-6-nonenoic acid (32.76 g, 88% yield, Z/E 11:1 by NMR analysis) as colorless oil product.

### Example 5

### Preparation of 8-methyl Nonanoic Acid

32 g of (Z)-8-methyl-6-nonenoic acid was dissolved in 150 ml of diisopropyl ether. 0.5 mol % of Pd/C powder was then dispersed in this solution. The mixture was hydrogenated with H₂ balloon at room temperature overnight. The catalyst was recovered by filtration. The solvent was recovered by distillation. 8-methyl nonanoic acid was obtained as colorless oil with >99% yield.

### Example 6

### Preparation of (E)-8-methyl-6-nonenoic Acid

In a 1 L two-necked round-bottom flask 100.00 g (6-Carboxyhexyl)triphenylphosphonium bromide and 49.07 g (2.0 equiv) KOtBu were dissolved in 300 mL 2-MeTHF under protection of nitrogen atmosphere and cooled using an ice water bath. The reaction mixture turned bright orange color while the compounds were dissolved. A solution of 18.92 g (1.2 equiv) isobutyraldehyde in 200 mL 2-MeTHF was slowly added to the cold reaction mixture, which quickly turned white. After the addition was completed, the reaction mixture was warmed to room temperature and stirred for 6 h. The reaction was quenched by addition of 500 ml H₂O. The MeTHF solvent was recovered by distillation. After cooling to room temperature, most of Ph₃PO was precipitated and collected as white powder by filtration. The filtrate was acidified with concentrated HCl to pH 2, the formed organic layer was collected, and the water phase was extracted with DIPE (100 mL×2). The organic phases were combined and concentrated. This crude intermediate was then treated with concentrated HNO₃ (0.03 equiv) at 85° C. under protection of nitrogen atmosphere for 24 hours. After cooling, the mixture was washed with water (50 mL×2). The aqueous phases were combined and extracted with DIPE (50 mL×2). The organic phases were combined, dried with anhydrous Na₂SO₄, and concentrated to give 53.1 g crude product. This crude product was then distilled under reduced pressure to give (E)-8-methyl-6-nonenoic acid (30.2 g, 81% yield, E/Z 86:14 by NMR analysis) as colorless oil product.

Preparation of 8-methyl Nonanoic Acid Starting from Cyclohexanone and Isobutyraldehyde

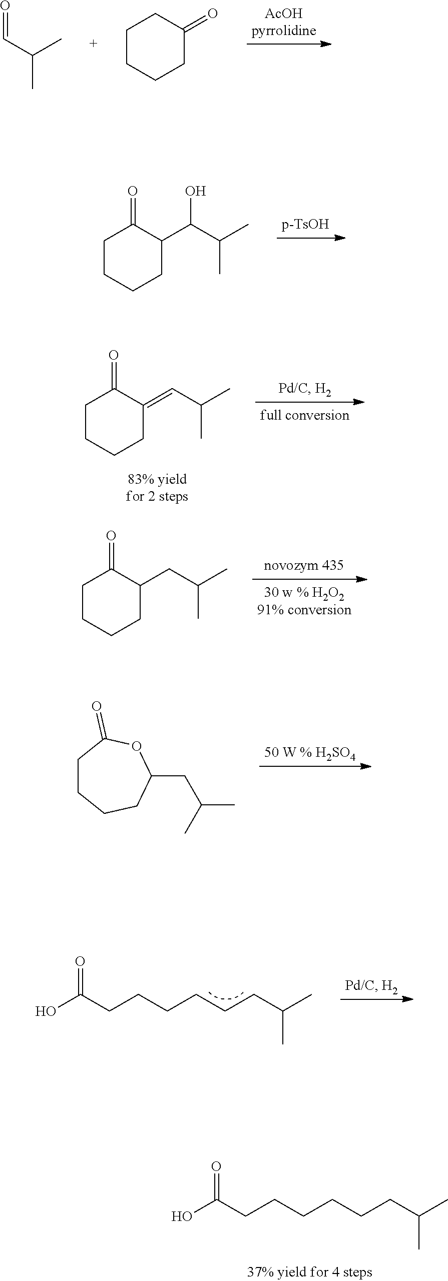

### Example 7

### Preparation of 2-(2-methylpropylidene)cyclohexan-1-one

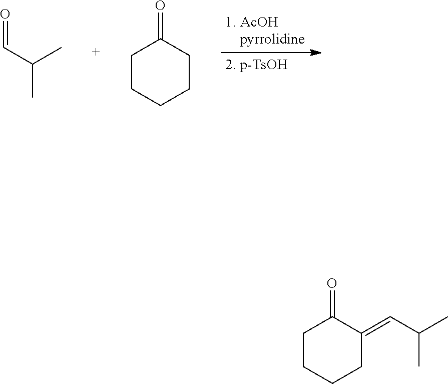

50 g isobutyraldehyde, 102 g cyclohexanone (1.5 equiv), 5 mol % of pyrrolidine and 5 mol % AcOH were heated and stirred at 40° C. for 12 h. After cooling to room temperature, the mixture was dispersed in 200 ml of water and 100 ml of toluene and organic phase was separated. The aqueous phase was extracted with toluene (50 ml×2). The organic phases were combined and treated with 4 mol % of p-TsOH·H2O catalyst at refluxing condition for 2 hours with a Dean-Stark trap to collect the generated water. After cooling again to room temperature, the acid was removed by washing with 30 ml of aqueous 1 M NaOH solution. Toluene and excess cyclohexanone were recovered by distillation. The enone product (87.6 g, 83% yield, light yellow) was then distilled out under reduced pressure.

### Example 8

### Preparation of 2-isobutylcyclohexanone

5 g of 2-(2-methylpropylidene)cyclohexan-1-one was dissolved in 10 mL of EtOAc. 0.2 mol % of Pd/C was added, and the hydrogenation was conducted at room temperature with H₂ balloon for 4 hours. Full conversion was achieved based on NMR analysis. The catalyst was recovered by filtration, and the filtrate was directly used in the following oxidation.

### Example 9

### Preparation of 7-isobutyloxepan-2-one

To the solution of 2-isobutylcyclohexanone in EtOAc, were added Novozym 435™ (250 mg) and 30% aqueous H₂O₂ (3 equiv). The mixture was stirred and heated at 50° C. After 24 h, 91% conversion was achieved based on NMR analysis. After cooling to room temperature, the lipase catalyst was recovered by filtration. The filtrate was washed with 5% aqueous Na₂S₂O₃ solution and brine to remove excess peroxide. The organic phase was concentrated to give the crude lactone.

### Example 10

### Preparation of 8-methyl-6-nonanoic Acid

The above crude lactone was dispersed in 5 M H₂SO₄ (40 mL) and heated at 110° C. oil bath. After 20 h, the mixture was cooled to room temperature, extracted with DIPE (20 mL×3). The organic phases were washed with brine, dried over anhydrous Na₂SO₄ and filtered. To the filtrate, 0.5 mol % Pd/C powder was added, and the hydrogenation was conducted at room temperature with H₂ balloon for 24 hours. After filtration to recover the Pd catalyst, the filtrate was concentrated and purified by flash chromatography on silica gel to give the 8-methyl-6-nonanoic acid (2.1 g, 37% yield from 5 g of the enone product of Example 7).

### Preparation of Capsaicinoids

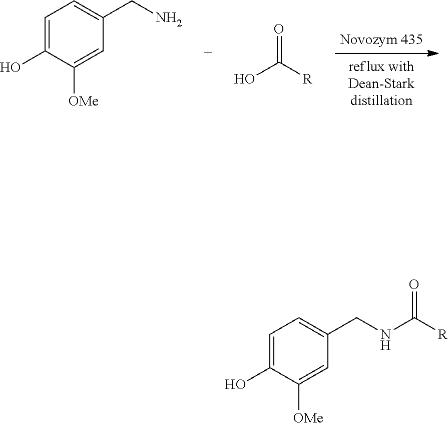

### Example 11

Preparation of capsaicin with excess amine in DIPE. At normal atmospheric pressure (approximately 1 atm), 8-methyl-6-nonenoic acid (3.65 g), vanillyl amine (1.1 equiv.), Novozym 435™ on beads (498.8 mg, 14 w/w % E/S) were refluxed (about 69° C.) in diisopropyl ether (45 mL) with a Dean-Stark trap to collect the generated water. After stirring at about 300 rpm overnight (19 h), the mixture was filtered to recover the lipase catalyst, and the filtrate was washed with 0.5 M aqueous HCl solution (10 mL). The aqueous phase was extracted with Et₂P (10 mL×2) and the organic phase were combined, dried over anhydrous Na₂SO₄ and concentrated to give 6.53 g product (99.7% yield, very pale yellow color).

### Example 12

Preparation of nonivamide with excess fatty acid in toluene. At normal atmospheric pressure (approximately 1 atm), Vanillylamine (4.89 g, 2.00 wt % water), nonanoic acid (1.01 equiv.) and Novozym 435™ on beads (1 g, 20 w/w % E/S) were refluxed (about 110° C.) in toluene (50 mL) with a Dean-Stark trap to collect the generated water. After stirring at about 300 rpm overnight (16 h), the conversion of nonanoic acid was >99%. After filtration to recover enzyme catalyst, the mixture was concentrated to give 9.14 g of product (99.6% yield, white color).

### Example 13

Preparation of nonivamide with excess fatty acid in cyclohexane. At normal atmospheric pressure (approximately 1 atm), Vanillyl amine (4.89 g, 2.00 wt % water), nonanoic acid (1.01 equiv.) and Novozym 435™ on beads (1 g, 20 w/w % E/S) were refluxed (about 81° C.) in cyclohexane (50 mL) with a Dean-Stark trap to collect the generated water. After stirring at about 300 rpm overnight (16 h), the conversion of nonanoic acid was >99%. After filtration to recover enzyme catalyst, the mixture was concentrated to give 9.10 g of product (99.1% yield, pale yellow color).

Because the use of lipase on beads is not feasible for large scale production due to costs for work-up, like filtering the lipase, etc., next experiment was performed using lipase immobilized on a rotary bed reactor.

### Example 14

Preparation of dihydrocapsaicin in fix-bed reactor. At normal atmospheric pressure (approximately 1 atm), In a 1 L reactor equipped with a rotating fix-bed filled with 12 g of Novozym 435™ (45-60 w/w % E/S), vanillylamine, slightly excess 8-methyl nonanoic acid (1.01 equiv), and diisopropyl ether (600 mL) were refluxed (about 69° C.) with a Dean-Stark trap to collect the generated water (FIG. 1). During reaction, the rpm was fixed at about 300 rpm.

After reaction, the hot solution was released out and cooled to room temperature. The dihydrocapsaicin product crystallized and was collected by filtration. The filtrate was directly recycled as solvent for more batches. The results are shown in Table 1. The yield of dihydrocapsaicin was 99.7% in average.

TABLE 1
Preparation of dihydrocapsaicin in fix-bed reactor

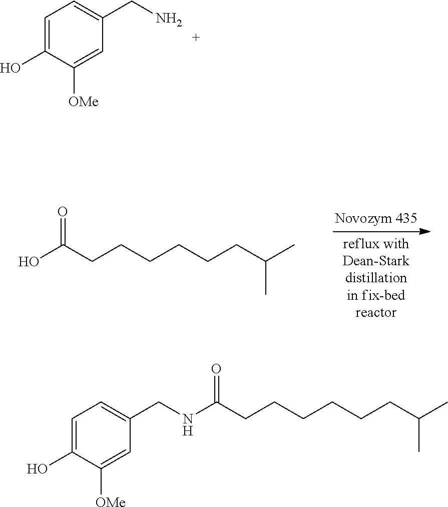 [SMILES: COc1cc(CN)ccc1O ; CC(C)CCCCCCC(=O)O ; COc1cc(CNC(=O)CCCCCCC(C)C)ccc1O]

| | Amine | moisture content of | Reaction time | Conversion | Yield |
|---|---|---|---|---|---|
| Cycle | (g) | amine (wt %) | (h) | (%) | (%) |
| 1 | 29.41 | 10.18 | 24 | >99 | |
| 2 | 34.34 | 23.08 | 24 | >99 | 99.8 |
| 3 | 31.80 | 16.95 | 24 | >99 | 100 |
| 4 | 31.80 | 16.95 | 24 | >99 | |
| 5 | 31.80 | 16.95 | 24 | >99 | |
| 6 | 24.89 | 16.95 | 16 | >99 | |

The results show good conversion rates and yields even after 6 cycles.

### Example 15

Preparation of capsaicin in fix-bed reactor (45w/w % E/S). The reactor system of Example 14 was cleaned by refluxing with DIPE solvent to remove residual dihydrocapsaicin. In this reactor, at normal atmospheric pressure (approximately 1 atm), vanillylamine, slightly excess 8-methyl-6-nonenoic acid (1.01 equiv), and diisopropyl ether (600 mL) were refluxed (about 69° C.) with a Dean-Stark trap to collect the generated water. During reaction, the rpm was fixed at about 300 rpm. After reaction, the hot solution was released out and cooled to room temperature. The capsaicin product crystallized and was collected by filtration. The filtrate was directly recycled as solvent for more batches. The results are shown in Table 2. The yield of capsaicin was 99.4% in average.

TABLE 2
Preparation of capsaicin in fix-bed reactor.

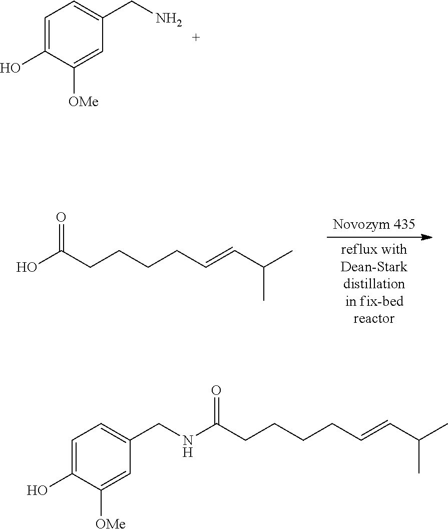 [SMILES: COc1cc(CN)ccc1O ; CC(C)/C=C/CCCCC(=O)O ; COc1cc(CNC(=O)CCCC/C=C/C(C)C)ccc1O]

| | Amine | moisture content of | Reaction time | Conversion | Yield |
|---|---|---|---|---|---|
| Cycle | (g) | amine (wt %) | (h) | (%) | (%) |
| 1 | 32.18 | 16.95 | 24 | >99 | |
| 2 | 32.18 | 16.95 | 24 | >99 | 99.5 |
| 3 | 32.18 | 16.95 | 24 | >99 | 99.9 |
| 4 | 27.83 | 3.90 | 20 | >99 | |
| 5 | 27.83 | 3.90 | 20 | >99 | |

The results show good conversion rates and yields even after 5 cycles.

### Example 16a

Preparation of nonivamide in fix-bed reactor (15-21 w/w % E/S). The reactor system of Example 15 was cleaned by refluxing with DIPE solvent to remove residual capsaicin. In this reactor, at normal atmospheric pressure (approximately 1 atm), vanillylamine, slightly excess nonanoic acid (1.01 equiv), and diisopropyl ether (600 mL) were refluxed (about 69° C.) with a Dean-Stark trap to collect the generated water. During reaction, the rpm was fixed at about 300 rpm. After reaction, the hot solution was released out and cooled to room temperature. The nonivamide product crystallized and was collected by filtration. The filtrate was directly recycled as solvent for more batches. The results are shown in Table 3a. The yield of nonivamide was 99.8% in average.

TABLE 3a
Preparation of nonivamide in fix-bed reactor.

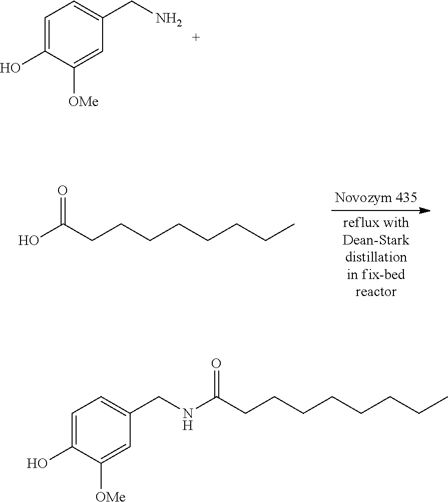 [SMILES: COc1cc(CN)ccc1O ; CCCCCCCCC(=O)O ; CCCCCCCCC(=O)NCc1ccc(O)c(OC)c1]

| | Amine | moisture content of amine | Reaction time | Conversion |
|---|---|---|---|---|
| Cycle | (g) | (wt %) | (h) | (%) |
| 1 | 39.89 | 3.90 | 24 | >99 |
| 2 | 39.89 | 3.90 | 24 | >99 |
| 3 | 44.88 | 3.90 | 24 | >99 |
| 4 | 45.48 | 5.16 | 24 | >99 |
| 5 | 45.48 | 5.16 | 24 | >99 |
| 6 | 45.48 | 5.16 | 24 | >99 |
| 7 | 50.53 | 5.16 | 24 | >99 |
| 8 | 48.90 | 2.00 | 24 | >99 |
| 9 | 58.68 | 2.00 | 24 | >99 |
| 10 | 58.68 | 2.00 | 24 | >99 |
| 11 | 78.24 | 2.00 | 24 | >99 |

The results show good conversion rates and yields even after 11 cycles.

### Example 16b

Preparation of nonivamide in 100 L fix-bed reactor. At normal atmospheric pressure (approximately 1 atm), in a 100 L reactor equipped with a rotating fix-bed filled with 1 kg of Novozym 435™ (50-100 w/w % E/S), vanillylamine, excess 8-methyl nonanoic acid (1.03 equiv), and diisopropyl ether (90 L) were refluxed (about 69° C.) with a Dean-Stark trap to collect the generated water. During reaction, the rpm was fixed at about 250 rpm. After reaction, the hot solution was released out and cooled to 15° C. The nonivamide product crystallized and was collected by filtration. The filtrate was directly recycled as solvent for more batches. The results are shown in Table 3b. The results show that the process of the invention can be used for large scale production of amides.

TABLE 3b
Preparation of nonivamide in 100 L fix-bed reactor

| | | moisture content | Reaction | |
|---|---|---|---|---|
| Cycle | Amine (g) | of amine (wt %) | time (h) | yield (%) |
| 1 | 1014 | 5.51 | 15 | 96.4 |
| 2 | 1992 | 2.66 | 24 | 95.6 |
| 3 | 1936 | <0.5 | 24 | 95.1 |

The results show good conversion rates and yields even after 3 cycles when the process is used at large scale.

### COMPARATIVE EXAMPLES

Preparation of Capsaicin from Fatty Acid with Drying Agents

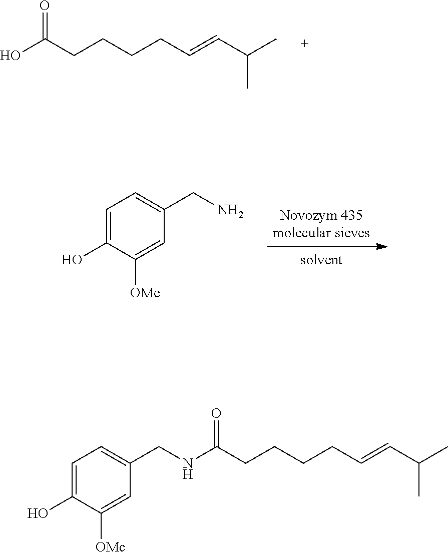

### Example 17

To a 200 mL reactor, were added in toluene (150 mL), 4 Å molecular sieves (10 g), immobilized enzyme (Novozym 435™, 0.59 g), 8-methyl-6-nonenoic acid (2.02 g), and vanillylamine (2 equiv). The reaction was conducted at 80° C. and monitored by NMR. After 6 hours, the conversion of acid was >99%. After filtration, the organic filtrate was cooled, successively washed with 1 M HCl (20 mL×2), water (20 mL×2), and brine (20 mL). After drying with anhydrous Na₂SO₄, the solvent was removed under vacuum. 3.07 g (84.7% yield) of capsaicin was obtained.

### Example 18

To a 25 mL flask, were added in t-BuOH (8 mL), 4 Å molecular sieves (600 mg), immobilized enzyme (Novozym 435™, 75 mg), 8-methyl-6-nonenoic acid (341 g), and vanillylamine (1.06 equiv). The reaction was conducted at 80° C. and monitored by NMR. After 10 hours, the conversion of acid was about 95%.

Preparation of Capsaicin with Ester as Acyl Donor:

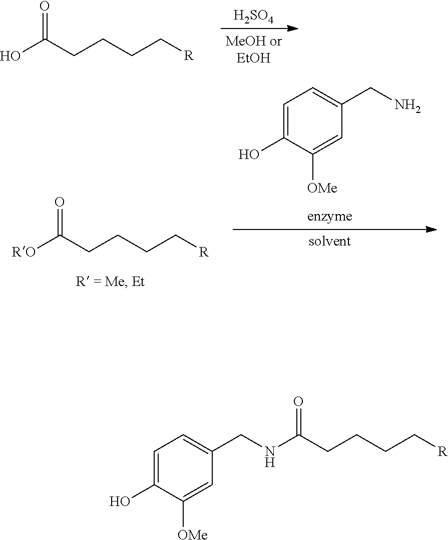

### Example 19

Preparation of methyl ester. 4.73 g of 8-methyl-6-nonenoic acid was dissolved in 30 ml of MeOH. To this solution, 5 drops of concentrated H₂SO₄ was added as catalyst. The resulting solution was refluxed overnight. After cooling to room temperature, most of MeOH was removed by rotary evaporator and the residue was dissolved in Et₂O (30 mL) and washed with 5% Na₂CO₃ solution (10 mL×2). The organic phase was dried with anhydrous Na₂SO₄ and concentrated to give the ester product with >99% yield.

### Example 20

Preparation of capsaicin with ester in fix-bed reactor. In a 1 L reactor equipped with a rotating fix-bed filled with 6 g of Novozym 435™ (10-20 w/w % E/S), ethyl ester of 8-methyl 6-nonenoic acid, excess vanillylamine (1.1 equiv), and diisopropyl ether (600 mL) were refluxed. After reaction, the hot solution was released out and cooled to room temperature. The solution was successively washed with 0.5 M HCl (60 mL), water (60 mL), and brine (20 mL). After drying with anhydrous Na₂SO₄, the solvent was recovered by rotary evaporation. The capsaicin product was obtained with light yellow color. The results are shown in Table 4.

TABLE 4
Preparation of capsaicin with ester in fix-bed reactor.

| Cycle | Ethyl ester (g) | Time (h) | Conversion (%) | Yield (%) |
|---|---|---|---|---|
| 1 | 30 | 22 | >99 | 92 |
| 2 | 30 | 16 | >99 | 93 |
| 3 | 60 | 40 | 90 | 80 |
| 4 | 30 | 42 | >99 | 93 |

### Example 21

Preparation of capsaicin from ester with distillation apparatus. To a flask equipped with short-path distillation apparatus, 923 mg of methyl ester of 8-methyl-6-nonenoic acid, 200 mg of Novozym 435™ on beads and 1.1 equiv. of vanillylamine were heated at 80° C. in 20 ml of t-BuOH. After 20 hours, the conversion of ester was >99% according to NMR analysis.

The results show that using esters is possible, but that the yield is lower compared to earlier examples 14, 15 and 16. An extra process step is needed because the methyl ester is prepared from the corresponding acid. Further, the work-up is tedious when up-scaling. Besides, recycling of the solvent is difficult, which is important for reducing costs and environmental impact of the process at large scale production.

### Example 22

Preparation of capsaicinoids in neat condition (no solvent). Experimental results are shown in Table 5.

TABLE 5
Preparation of capsaicinoids in neat condition.

| | | | Lipase | | | | |
|---|---|---|---|---|---|---|---|
| | Vanillylamine | Acyl donor | (g/g | Temp | Time | | Yield |
| Entry | (equiv) | (equiv) | amine) | (° C.) | (h) | product | (%) |
| 1* | 1 | nonanoic | 0.2 | 100 | 18 | nonivamide | 37 |
| | | acid | | | | | |
| | | (3 equiv) | | | | | |
| 2 | 1 | ethyl 8- | 0.2 | 80 | 16 | capsaicin | 91 |
| | | methyl-6- | | | | | |
| | | nonenoate | | | | | |
| | | (2 equiv) | | | | | |
| 3 | 2 | 8-methyl-6- | — | 150 | 16 | capsaicin | 37 |
| | | nonenoic | | | | | |
| | | acid | | | | | |
| | | (1 equiv) | | | | | |
| 4 | 1.2 | methyl 8- | — | 150 | 16 | capsaicin | 10 |
| | | methyl-6- | | | | | |
| | | nonenoate | | | | | |
| | | (1 equiv) | | | | | |

*under vacuum.

The results show that the yield of the process is decreased using reduced pressure (Entry 1).

### Example 23

### Preparation of Capsaicin With Lipase Catalyst and Without Dehydration

Vanillylamine (1 mmol), 8-methyl-6-nonenoic acid (1 mmol), and Novozym 435™ (45 mg) were stirred in toluene (4 mL) and heated at 80° C. for 48 h. 72% conversion was achieved based on NMR analysis.

### Example 24

### Preparation of Capsaicin Without Lipase Catalyst and With Dean-Stark Distillation

With a Dean-Stark trap to collect generated water, vanillylamine (1 mmol) and 8-methyl-6-nonenoic acid (1 mmol) were refluxed in toluene (4 mL) at 115° C. oil bath for 20 h. 19% conversion was achieved based on NMR analysis.

### Example 25

### Preparation of Capsaicin With Acid Chloride as Acyl Donor

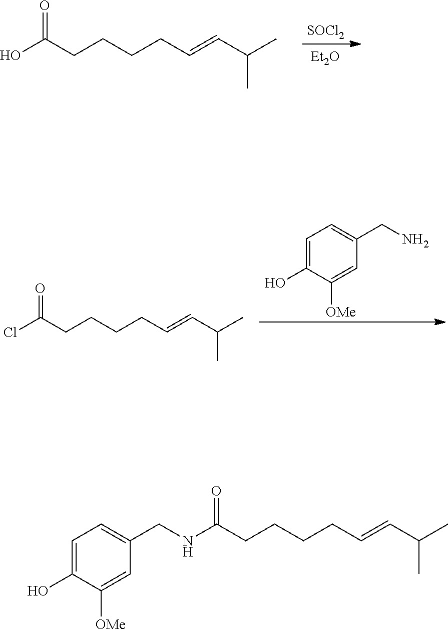

In one 1 L flask, 47.17 g of 8-methyl-6-nonenoic acid was dissolved in 400 ml of anhydrous Et₂O. 30.1 mL of SOCl₂ (1.5 equiv) was dissolved in 100 ml of anhydrous Et₂O, and slowly added to the acid solution. The resulting solution was refluxing for 3 hours, and then the excess SOCl₂ and solvent were removed under reduced pressure. The resulting acid chloride was then dissolved in 200 ml of anhydrous Et₂O. To a slurry of 84.7 g vanillylamine (2 equiv) in 400 ml of anhydrous Et₂O, was added slowly the acid chloride solution in 2 hours. After addition, the refluxing was continued for 2 hours. The mixture was cooled with ice water bath, the precipitate was filtered. The organic filtrate was successively washed with 1 M HCl (50 mL×2), water (50 mL×2), and brine (50 mL). After drying with anhydrous Na₂SO₄, the solvent was removed under vacuum. 54.3 g (64.2% yield) of capsaicin was obtained.

### Preparation of Miscellaneous Amides by the Combination Of Enzymatic Catalysis and Dean-Stark Distillation

Preparation of (R)-2-methoxy-N-(1-phenylethyl)acetamide

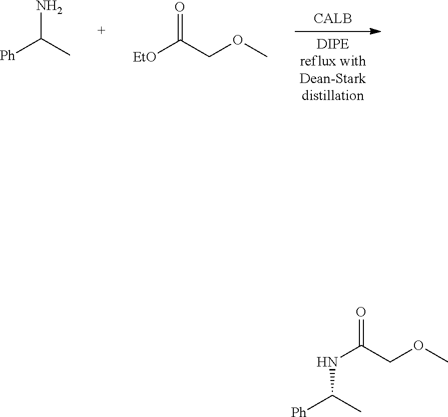

### Example 26

With a Dean-Stark trap to collect generated water, 1-phenylethan-1-amine (1 mmol), ethyl 2-methoxyacetate (2 mmol), and Novozym 435™ on beads (45 mg) were refluxed in diisopropryl ether (30 mL) at 90° C. oil bath for 10 h. After work up, (R)-2-methoxy-N-(1-phenylethyl)acetamide was obtained with 85% yield and 8% ee.

### Example 27

With a Dean-Stark trap to collect generated water, 1-phenylethan-1-amine (1 mmol), ethyl 2-methoxyacetate (2 mmol), and Novozym 435(tm) on beads (45 mg) were refluxed in diisopropryl ether (30 mL) at 90° C. oil bath for 3 h. After work up, (R)-2-methoxy-N-(1-phenylethyl)acetamide was obtained with 75% yield and 55% ee.

### Example 28

With a Dean-Stark trap to collect generated water, 1-phenylethan-1-amine (1 mmol), ethyl 2-methoxyacetate (2 mmol), and Novozym 435™ on beads (45 mg) were refluxed in diisopropryl ether (30 mL) at 90° C. oil bath for 1 h. After work up, (R)-2-methoxy-N-(1-phenylethyl)acetamide was obtained with 48% yield and 97% ee.

### Example 29

### Preparation of (R)-2-methoxy-N-(1-(4-methoxyphenyl)ethyl)acetamide.

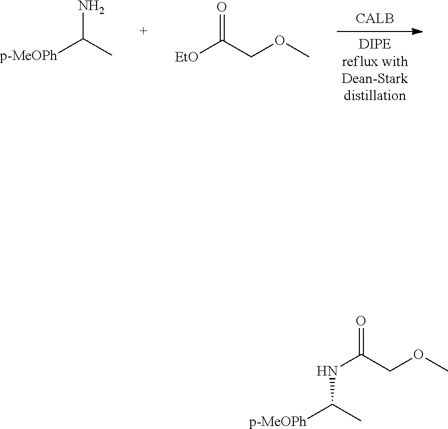

With a Dean-Stark trap to collect generated water, 1-(4-methoxyphenyl)ethan-1-amine (1 mmol), ethyl 2-methoxyacetate (2 mmol), and Novozym 435™ on beads (45 mg) were refluxed in diisopropryl ether (30 mL) at 90° C. oil bath for 0.83 h. After work up, (R)-2-methoxy-N-(1-(4-methoxyphenyl)ethyl)acetamide was obtained with 44% yield and 97% ee.

### Example 30

### Preparation of N-phenethylnonanamide

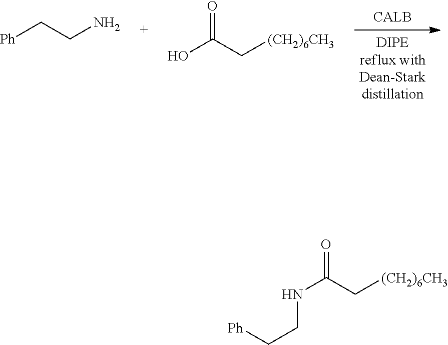

With a Dean-Stark trap to collect generated water, 2-phenylethan-1-amine (1 mmol), nonanoic acid (1.05 mmol), and Novozym 435™ on beads (45 mg) were refluxed in diisopropryl ether (30 mL) at 90° C. oil bath for 10 h. After work up, N-phenethylnonanamide was obtained with 98% yield.

### Example 31

### Preparation of N-phenethylstearamide

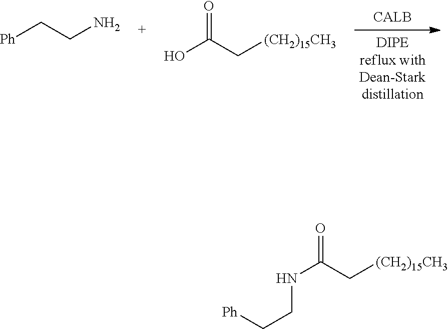

With a Dean-Stark trap to collect generated water, 2-phenylethan-1-amine (1 mmol), stearic acid (1.05 mmol), and Novozym 435™ on beads (45 mg) were refluxed in diisopropryl ether (30 mL) at 90° C. oil bath for 10 h. After work up, N-phenethylstearamide was obtained with 98% yield.

The present invention is not limited to the embodiments disclosed but may be varied and modified within the scope of the following claims.

1. A process for enzymatic synthesis of amides of formula from amines of formula I and compounds of formula II, 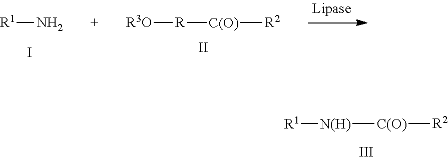
wherein R¹ is selected from the group comprising C₅₋₁₂aryl-, and C₅₋₁₂aryl-C₁₋₆alkyl-,
which R¹ may optionally be substituted with one or more substituent selected from the group comprising hydrogen, hydroxy and C₁₋₆alkoxy-, and
wherein R² is selected from the group comprising hydrogen, C₁₋₃₀alkyl-, C₁₋₃₀alkenyl-,
wherein R³ is selected from the group comprising hydrogen, and C₁₋₆alkyl-,
wherein R is a bond or C₁₋₆alkyl-,
characterized in that the lipase is immobilized on a rotary bed reactor or on a spin-fixed-bed reactor and a Dean-Stark apparatus is used for dehydration.

2. The process according to claim 1, wherein R¹ is selected from the group comprising C₆₋₇aryl-, and C₅₋₇aryl-C₁₋₃alkyl-,
which R¹ may optionally be substituted with one or more substituent selected from the group comprising hydrogen, hydroxy, and C₁₋₃alkoxy-, and
wherein R² is selected from the group comprising hydrogen, C₅₋₁₅alkyl-, C₅₋₁₅alkenyl-, C₅₋₁₅alkoxy-, and C₅₋₁₅alkyl-O-C₁₋₆alkyl-,
wherein R³ is selected from the group comprising hydrogen, and C₁₋₃alkyl-, and
wherein R is a bond or C₁₋₃alkyl-.

3. The process according to claim 1,
wherein R¹ is C₅₋₇aryl-C₁₋₃alkyl-,
which R¹ may optionally be substituted with one or more substituent selected from the group comprising hydrogen, hydroxy and C₁₋₃alkoxy-,
wherein R² is selected from the group comprising C₅₋₁₆alkyl- and C₅₋₁₅alkenyl-, and
wherein R³ is hydrogen, methyl or ethyl, and
wherein R is a bond.

4. The process according to claim 1, wherein compounds of formula III are compounds of formula IV 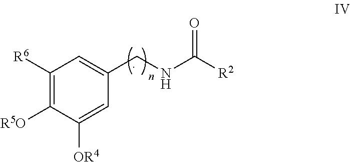
wherein n is 1 or 2,
wherein R² is selected from the group comprising hydrogen, C₃₋₃₀alkyl-, and C₃₋₃₀alkenyl-,
wherein R⁴ or R⁵ is selected from the group comprising hydrogen, and C₁₋₆alkyl-,
wherein R⁶ is selected from the group comprising hydrogen, hydroxy, oxy, halogen, carboxy, amine, amide, C₁₋₁₀alkyl-, C₂₋₁₀alkenyl-, C₂₋₁₀alkynyl-, C₃₋₁₂cycloalkyl-, C₃₋₁₂cycloalkenyl- and C₅₋₁₂aryl-,
which R⁶ may optionally be substituted with one or more substituent selected from the group comprising hydrogen, hydroxy, and C₁₋₆alkoxy-.

5. The process according to claim 4, wherein compounds of formula III are compounds of formula IV 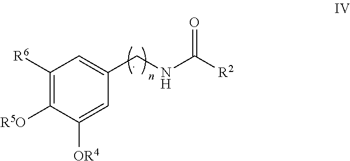
wherein n is 1 or 2,
wherein R² is selected from the group comprising C₃₋₁₈alkyl- and C₃₋₁₈alkenyl-,
wherein R⁴ or R⁵ is selected from the group comprising hydrogen, C₁₋₆alkyl-, and
R⁶ is hydrogen.

6. The process according to claim 4, wherein compounds of formula III are compounds of formula IV 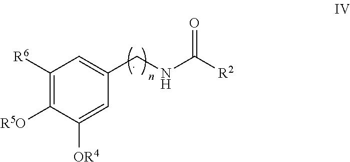
wherein n is 1 or 2,
wherein R² is selected from the group comprising C₅₋₁₆alkyl- and C₅₋₁₅alkenyl-,
wherein R⁴ or R⁵ is selected from the group comprising hydrogen, C₁₋₃alkyl-, and
R⁶ is hydrogen.

7. The process according to claim 1, for enzymatic synthesis of amides of formula III from amines of formula I and compounds of formula IIa, 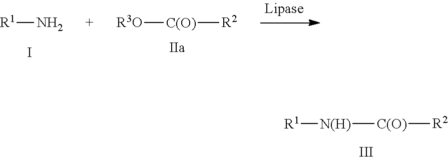
wherein R¹ is selected from the group comprising C₅₋₁₂aryl-, and C₅₋₁₂aryl-C₁₋₆alkyl-,
which R¹ may optionally be substituted with one or more substituent selected from the group comprising hydrogen, hydroxy, and C₁₋₆alkoxy-, and
wherein R² is selected from the group comprising hydrogen, C₁₋₃₀alkyl-, C₁₋₃₀alkenyl-,
wherein R³ is selected from the group comprising hydrogen and C₁₋₆alkyl-,
characterized in that
the lipase is immobilized on a rotary bed reactor or on a spin-fixed-bed reactor and a Dean-Stark apparatus is used for dehydration.

8. The process according to claim 7, wherein R¹ is C₅₋₇aryl-C₁₋₃alkyl-,
which R¹ may optionally be substituted with one or more substituent selected from the group comprising hydrogen, hydroxy and C₁₋₃alkoxy-, and
wherein R² is C₅₋₁₅alkyl- and C₅₋₁₅alkenyl-, and
wherein R³ is hydrogen, methyl or ethyl.

9. The process according to claim 1, wherein no solvent is used, or the solvent is an organic solvent selected from the group comprising methyl tert-butyl ether, diisopropylether, C₁₋₆alkyl-O-C₁₋₆alkyl ethers, hexane and other C₅₋₁₀alkanes, cyclohexane and other C₅₋₁₀cycloalkanes, benzene, toluene, xylene, tert-butanol, tert amyl alcohol, other bulky secondary or tertiary C₅₋₁₀ alcohols and any esters thereof, or mixtures thereof.

10. The process according to claim 1, wherein no solvent is used, or the solvent is an organic solvent selected from the group comprising diisopropylether, cyclohexane, toluene or tert-butanol, or mixtures thereof.

11. The process according to claim 1, wherein the lipase is selected from the group comprising Candida antarctica lipase A, Candida antarctica lipase B, cross-linked Substilisin A protease, Porcine pancreas lipase, Candida cylindracea lipase, Rhizopus arrhizus, Penicillum cyclopium, Mucor miehei, Thermomyces lanuginosus lipase, Candida rugosa lipase and Pseudomonas lipoprotein lipase.

12. The process according to anyone of claim 1, wherein the lipase is Candida antarctica lipase.

13. The process according to claim 1, wherein the process temperature is between room temperature and 150° C. and the pressure is between 0.900 0.090 and 0.200 MPa, or about 0.1 MPa.

14. The process according claim 1, wherein the rotary bed reactor is loaded for 10 to 75wt % with the lipase.

15. A process according to claim 1, wherein compounds of formula II, wherein R² is a C₆₋₁₈alkyl or C₆₋₁₈alkenyl, which may be straight or branched, are prepared comprising the steps of 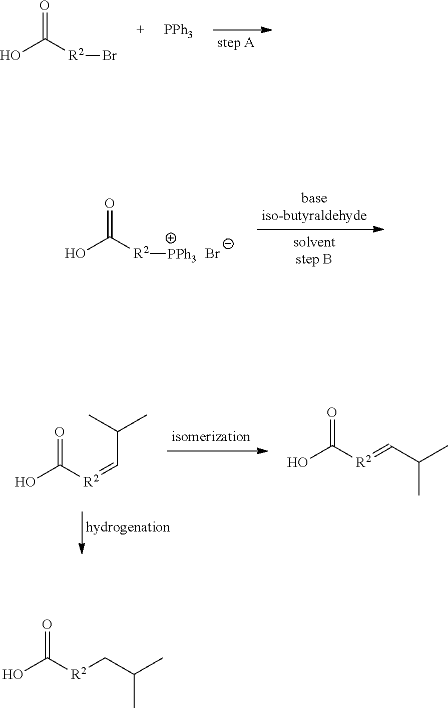
step A-1, wherein the reaction is performed without solvent or with an organic solvent,
step B-1, wherein a solvent is an aprotic organic solvent, and
step B-1, wherein a base a sodium or potassium alkoxides,
optionally isomerization step C-1, wherein a catalyst is selected from the group comprising HNO₂, HNO₃ and combinations of NaNO₂/HNO₃, NaNO₂/NaNO₃/H₂SO₄, that can generate HNO₂ or HNO₃, and
hydrogenation step D-1, wherein a catalyst is a heterogeneous hydrogenation catalyst and a hydrogen source is hydrogen gas.

16. The process according to claim 15, wherein the organic solvent in step A-1 is ethyl acetate,
wherein the aprotic organic solvent in step B-1 is selected from the group comprising 2-methyl tetrahydrofuran, tetrahydrofuran and toluene,
wherein the sodium or potassium alkoxide base in step B-1 is selected from the group comprising NaH, KH, t-BuOK, t-BuONa, and
wherein the heterogeneous hydrogenation catalyst in hydrogenation step D-1 is selected from the group comprising Pd/C and Pd/Al₂O₃.

17. A process according to claim 1, wherein compounds of formula II, wherein R² is 8-methyl-nonanyl, are prepared comprising the steps of 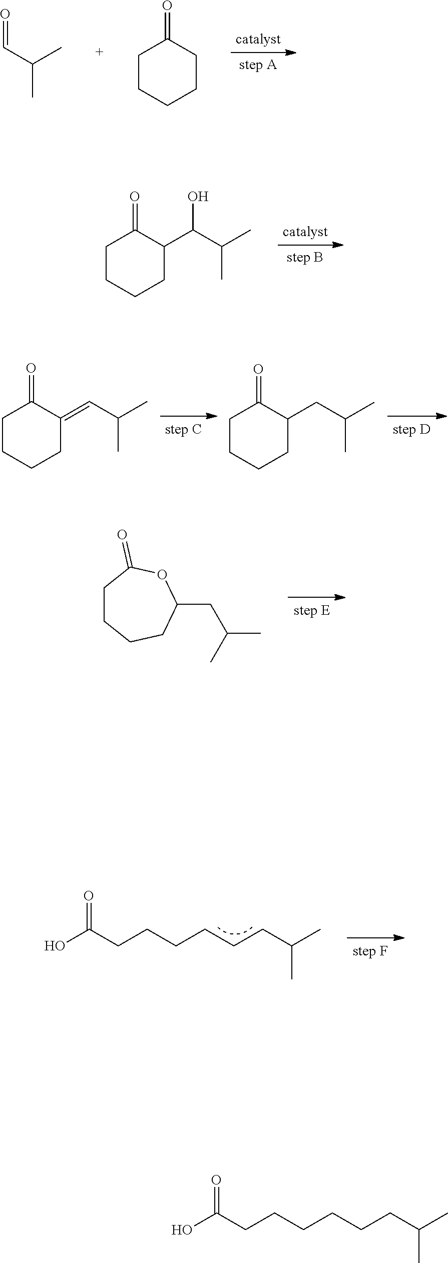
step A-2, wherein the reaction is performed without solvent or with any organic solvent and a catalyst is selected from the group comprising amines and inorganic bases,
step B-2, wherein the reaction is performed without solvent or with an organic solvent, and a catalyst is an acid,
step C-2, wherein a catalyst is a heterogeneous hydrogenation catalyst and a hydrogen source is hydrogen gas,
step D-2, wherein an oxidant is a peroxide and a catalyst is a lipase, and
step E-2, wherein a reaction medium is an acidic media, and
Step F-2, wherein a catalyst is a heterogeneous hydrogenation catalyst and a hydrogen source is hydrogen gas.

18. The process according to claim 17, wherein the organic solvent in step A-2 is selected from the group comprising toluene, and a catalyst is selected from the group comprising pyrrolidine and corresponding salts, NaOH and KOH,
wherein the organic solvent in step B-2 is selected from the group comprising toluene, and the acid is selected from the group comprising p-TsOH, sulfuric acid and Amberlyst-15,
wherein the catalyst in step C-2 is selected from the group comprising Pd/C, Pd/Al₂O₃,
wherein the oxidant in step D-2 is selected from the group comprising aqueous H₂O₂ and peroxy acids and the lipase is selected from the group comprising Candida antarctica lipase A, Candida antarctica lipase B, cross-linked Substilisin A protease, Porcine pancreas lipase, Candida cylindracea lipase, Rhizopus arrhizus, Penicillum cyclopium, Mucor miehei, Thermomyces lanuginosus lipase, Candida rugosa lipase and Pseudomonas lipoprotein lipase, and
wherein the reaction medium in step E-2 is selected from the group comprising aqueous sulfuric acid solution, and
wherein the catalyst in step F-2 is selected from the group comprising Pd/C, Pd/Al₂O₃, Pd/molecular sieves, Pt/C, Pt/Al₂O₃, and Pt/molecular sieves.

19. The processes according to any one of claim 1, for large scale production (>1 kg) of compounds of formula III.

20. The process according to anyone of claim 1, wherein the process is performed at atmospheric pressure and at temperatures below 100° C.
